(19) United States
(12) Patent Application Publication
(10) Pub. No.: US 2025/0250326 A1
(43) Pub. Date: Aug. 7, 2025

(54) PASSIVE IMMUNIZATION WITH ANTI-AAV NEUTRALIZING ANTIBODIES TO PREVENT OFF-TARGET TRANSDUCTION OF INTRATHECALLY DELIVERED AAV VECTORS

(71) Applicant: The Trustees of the University of Pennsylvania, Philadelphia, PA (US)

(72) Inventors: Christian Hinderer, Baltimore, MD (US); Makoto Horiuchi, Philadelphia, PA (US); James M. Wilson, Philadelphia, PA (US)

(73) Assignee: The Trustees of the University of Pennsylvania, Philadelphia, PA (US)

(21) Appl. No.: 18/853,007

(22) PCT Filed: Apr. 6, 2023
(86) PCT No.: PCT/US2023/065422

§ 371 Date: Sep. 30, 2024

Related U.S. Application Data
(60) Provisional application No. 63/328,227, filed on Apr. 6, 2022.

(51) Int. Cl. C07K 16/10; A61K 39/00

(52) U.S. Cl. CPC C07K 16/1003; A61K 2039/505; C07K 2317/24

(57) ABSTRACT

Provided herein are compositions useful for preventing off-target effects of recombinant adeno-associated (AAV) gene therapy vectors administered to the central nervous system or to the eye. The compositions comprise anti-AAV neutralizing antibodies delivered via a systemic route prior to delivery of a recombinant AAV.

### BACKGROUND OF THE INVENTION

Many genetic disorders negatively impact the normal development and function of the central nervous system (CNS). Gene therapy using neurotropic adeno-associated virus (AAV) vectors such as AAV9 represent an emerging real-world solution for such diseases. Previously published studies showed that high-dose intravenous AAV administration causes severe toxicity in nonhuman primates (NHPs), characterized by thrombocytopenia and acute liver injury. [Hinderer, C., et al (2018). Severe Toxicity in Nonhuman Primates and Piglets Following High-Dose Intravenous Administration of an Adeno-Associated Virus Vector Expressing Human SMN. Hum Gene Ther 29, 285-298].

Studies have reported similar toxicity in the clinical setting: about 20% to 30% of patients treated with Zolgensma® [onasemnogene abeparvovec-xiol, a recombinant self-complementary AAV9 containing a transgene encoding the human survival motor neuron (SMN) protein, under the control of a cytomegalovirus enhancer/chicken-β-actin hybrid promote; Novartis, US] show liver-associated adverse events, with a few serious cases requiring additional treatment. [Chand, D., et al, (2021). Hepatotoxicity following administration of onasemnogene abeparvovec (AVXS-101) for the treatment of spinal muscular atrophy. J Hepatol 74, 560-566].

Intrathecal delivery of AAV vectors allows effective transduction of neurons, astrocytes, and ependymal cells, as the blood brain barrier is bypassed. More specifically, intra-cisterna magna (ICM) administration of AAV gene therapy enables more widespread transgene expression throughout the central nervous system (CNS) of non-human primates (NHPs) than lumbar puncture. [Hinderer, C., et. al., (2014). Widespread gene transfer in the central nervous system of cynomolgus macaques following delivery of AAV9 into the cisterna magna. Mol Ther Methods Clin Dev 1, 14051; Hinderer, C., et al., (2020). Translational Feasibility of Lumbar Puncture for Intrathecal AAV Administration. Mol Ther Methods Clin Dev 17, 969-974.

A significant level of liver transduction still occurs with ICM AAV administration, suggesting vector leakage from the intrathecal space in large animals. [Ballon, D. J., et al. (2020). Quantitative Whole-Body Imaging of I-124-Labeled Adeno-Associated Viral Vector Biodistribution in Nonhuman Primates. Hum Gene Ther 31, 1237-1259]. Thus, there remains a need to reduce off-target liver transduction to improve the safety of CNS-targeted AAV gene therapy.

Further, it has been previously published that gene transduction to the CNS was not affected by pre-existing anti-AAV neutralizing antibodies (NAbs), while liver transduction decreased significantly upon intrathecal vector administration in dogs and NHPs. [Gray, S. J., et al, (2013). Global CNS gene delivery and evasion of anti-AAV-neutralizing antibodies by intrathecal AAV administration in non-human primates. Gene Ther 20, 450-459; Hinderer. C., et al. (2018). Evaluation of Intrathecal Routes of Administration for Adeno-Associated Viral Vectors in Large Animals. Hum Gene Ther 29, 15-24].

What are needed are methods for preventing off-target effects of gene therapy vector delivery.

### SUMMARY OF THE INVENTION

The compositions and regimens provided herein minimize off-target effects of a viral vector delivery without negatively impacting the therapeutic efficacy of the viral vector by ablating the effect of neutralizing antibodies to a selected viral vector capsid. In certain embodiments, the viral vector is a rAAV and the patients are pre-treated intravenously (or via another systemic route) with an anti-AAV capsid neutralizing antibody (NAb) prior to administration of the administered to the central nervous system or to the eye.

In one aspect, a combination regimen is provided for preventing systemic uptake of a recombinant adeno-associated virus (AAV) vector delivered to the central nervous system (CNS) or intraocularly, the regimen comprising (a) pretreating the patient by systemically administering a composition comprising anti-AAV capsid neutralizing antibodies directed against an AAV capsid of the recombinant AAV vector, and (b) administering to the CNS or intraocularly a recombinant AAV vector comprising the AAV capsid and a vector genome comprising a nucleic acid sequence encoding a gene product operably linked to regulatory sequences that direct expression of the gene product in a target cell in the CNS (or a subset of CNS cells) or in the eye (or a subset of cells in the eye). In certain embodiments, the pretreating comprises intravenously administering the composition comprises polyclonal antibodies. In certain embodiments, the composition comprises pooled immunoglobulin from patients having high anti-AAV titers. In certain embodiments, the composition comprises at least one anti-AAV monoclonal antibody. In certain embodiments, the composition comprises a cocktail of anti-AAV monoclonal antibodies. In certain embodiments, the pretreating occurs at least two hours before and/or up to five days prior to administration of the recombinant AAV vector. In certain embodiments, the composition comprising the anti-AAV antibodies is administered intravenously. In certain embodiments, the recombinant AAV vector is administered intrathecally. In certain embodiments, the recombinant AAV vector is administered to the eye, optionally intravitreally, intra-retinally, subretinally, or suprachoroidally. Collectively, these may be termed “intraocular” injection.

In another aspect, a method is provided for increasing central nervous system transduction of a recombinant AAV-mediated gene therapy comprising: (a) systemically delivering a composition comprising anti-AAV neutralizing antibodies that bind an AAV capsid of a recombinant AAV vector, and (b) administering to the CNS of the patient a recombinant AAV vector that comprises the AAV capsid and a vector genome comprising a nucleic acid sequence encoding a gene product operably linked to regulatory sequences that direct expression of the gene product in a target cell in the CNS.

In another aspect, an anti-AAV pharmaceutical composition useful for pretreatment a recombinant AAV gene therapy patient is provided. The composition comprises a physiologically compatible aqueous suspension and anti-AAV neutralizing antibodies formulated for systemic delivery to a human patient. In certain embodiments, the anti-AAV neutralizing antibodies are in a dose of about 500 mg to about 2500 mg. In certain embodiments, the anti-AAV neutralizing antibodies comprise polyclonal antibodies. In certain embodiments, the composition comprises pooled immunoglobulin from subjects having high anti-AAV titers. In certain embodiments, the composition comprises at least one anti-AAV monoclonal antibody. In certain embodiments, the composition comprises a cocktail of anti-AAV monoclonal antibodies.

In yet another aspect, use of an anti-AAV pharmaceutical composition is provided which comprises (a) systemically passively immunizing a patient with the anti-AAV pharmaceutical composition, and (b) administering to the CNS or eye of the patient a recombinant AAV vector comprising an AAV capsid and a vector genome comprising a nucleic acid sequence encoding a gene product operably linked to regulatory sequences that direct expression of the gene product in a target cell in the CNS or in the eye.

In another aspect, an anti-AAV composition is provided which is to be administered systemically for passive immunization of a patient to reduce off-target transduction of the recombinant AAV in the CNS or in the eye.

These and other advantages of the invention will be readily apparent from the following detailed description of the invention.

### BRIEF DESCRIPTION OF THE DRAWINGS

FIG. 1A-FIG. 1C provide pharmacokinetics and protein biodistribution of transgene product 3D6 mouse monoclonal antibody in mice following intracerebroventricular (ICV) administration of AAVhu68.CB7.CI.3D6.rBG. Mice received the vector via unilateral ICV at indicated doses and expression of 3D6 in the brain homogenates (FIG. 1A) and serum (FIG. 1B) was measured by antigen-binding assay at 7, 14, 28, and 56-days post AAV administration. 3D6 was measured in homogenates of selected organs from mice treated with 1e10 GC dose at 56 days post AAV (FIG. 1C). Values are presented as mean+/−SEM.

FIG. 2A-FIG. 2J. FIG. 2A provides averaged AAV hu68 NAb titers in the reciprocal of serum dilution. Mice pre-treated with IVIG received AAV vector at 1 day after IVIG (IVIG (day 1)). Mice actively immunized with IM-AAV were injected with ICV-AAV.3D6 at 42 days after IM-AAV (IM-AAV (day 42)). Serum samples from naïve mice were used as controls. ** indicates p<0.01. (FIG. 2B-FIG. 2G) 3D6 expression in mouse tissue homogenates at 56 days post-ICV-vector. 3D6 antigen-binding assay data for the brain (FIG. 2B), serum (FIG. 2C), liver (FIG. 2D), heart (FIG. 2E), lung (FIG. 2F), and kidney (FIG. 2G) from mice with IVIG pre-treatment and then ICV-AAV.3D6. Those with IM-AAV, and then ICV-AAV.3D6, or only with ICV-AAV.3D6 are shown. Mouse tissue homogenates from untreated mice were used as negative controls. *, **, ***, and **** indicate p<0.05, p<0.01, p<0.005, and p<0.001, respectively, compared to the negative control. FIG. 2H-FIG. 2J provide 3D6 in situ hybridization (ISH) for mouse brain sections at 28 days post-ICV vector administration. Paraffin-embedded coronal brain sections from mice with IVIG pre-treatment and then ICV-AAV.3D6 (IVIG+ICV-vector), those with IM-AAV and then ICV-AAV.3D6 (IM-AAV+ICV-vector), or only with ICV-AAV.3D6 (ICV-vector) were hybridized with a fluorescent-labeled 3D6-specific probe. Nuclei were counterstained with 4′,6-diamidino-2-phenylindole (DAPI). Scale bars indicate 2 mm.

FIG. 3A-FIG. 3D provide gene transduction and transgene expression in NHPs treated with ICM-vector expressing 2.10A mAb with or without IVIG pre-treatment. Averaged 2.10A mAb concentrations in serum (FIG. 3A) and CSF (FIG. 3B) are shown along the course of the study. Vector genome copy numbers in the selected CNS tissue blocks and peripheral tissue blocks (FIG. 3C) were determined by quantitative PCR with a transgene-specific assay. 2.10A mAb mRNA was measured in selected CNS tissue blocks and peripheral tissue blocks (FIG. 3D) by quantitative PCR with transgene-specific assay. * indicates p<0.05.

FIG. 4A-FIG. 4B show area under curve (AUC) of serum (FIG. 4A) and CSF (FIG. 4B) 2.10A rhIgG1 expression along the NAb titer at day 0 as decimal numbers, where 1 was used for NAb titers below detection, <1:5. Dotted and solid lines indicate linear regression lines for ICM-vector and IVIG+ICM-vector groups, respectively. R² and non-zero p values from linear regression analysis are shown. * indicates p<0.05.

FIG. 5A-FIG. 5E show vector genome biodistribution data for individual NHPs for non-CNS tissues. FIG. 5A shows results for heart. FIG. 5B shows results for liver. FIG. 5C shows results for kidney. FIG. 5D shows results for muscle. FIG. 5E shows results for spleen. Tissue samples from necropsy at day 88 to 91 post-AAV administration were subjected to vector genome biodistribution qPCR analysis. Vector genome copy numbers for individual NHPs are shown along the NAb titer at day 0 as decimal numbers, where 1 was used for NAb titers below detection, <1:5. R² and non-zero p values from linear regression analysis are shown. * indicates p<0.05.

FIG. 6A-FIG. 6J provide vector genome biodistribution data for individual NHPs for CNS tissues, including frontal cortex (FIG. 6A), parietal cortex (FIG. 6B), temporal cortex (FIG. 6C), occipital cortex (FIG. 6D), hippocampus (FIG. 6E), medulla (FIG. 6F), cerebellum (FIG. 6G), cervical spinal cord (FIG. 6H), thoracic spinal cord (FIG. 6I), lumbar spinal cord (FIG. 6J). Tissue samples from necropsy at day 88 to 91 post-AAV administration were subjected to vector genome biodistribution qPCR analysis. Vector genome copy numbers for individual NHPs are shown along the NAb titer at day 0 as decimal numbers, where 1 was used for NAb titers below detection, <1:5. R² and non-zero p values from linear regression analyses are shown.

FIG. 7A-FIG. 7C show blood liver enzymes and DRG pathology scores in NHPs. The level of liver enzymes, ALT (FIG. 7A) and AST (FIG. 7B), in NHPs treated with ICM-vector only (RA2146, RA2335, RA2463, and RA1776) and those received IVIG+ICM vector (RA2393, RA2471, RA2476, and RA1825) are shown. FIG. 7C provides a summary of DRG pathology scores on DRG and spinal cord (SP) sections from the NHPs used in this study.

FIG. 8 illustrates the study in Example 2 investigating the use of plasma-derived, pooled human immunoglobulin (IG) as a source of anti-AAV neutralizing antibodies to reduce peripheral transduction resulting from vector leakage into systemic circulation. In this study, mice were pre-treated intravenously (IV) at 2 hours before recombinant AAV treatment (−2). IVIG was delivered to reduce serum levels of transgene expression, following which mice were administered with AAVrh91.test transgene at a dose of 1×10¹¹ GC/mouse via ICV injection. On day 28 mice were euthanized, whole body perfusion was performed, and brain and liver tissue samples were collected.

FIG. 9A-FIG. 9B show expression levels in collected tissues following ICV injection with and without IVIG, plotted as bioluminescence intensity in photons/see (luciferase) on day 7, 14, and 28. FIG. 9A shows expression levels in collected brain tissue. FIG. 9B shows expression levels in collected liver tissue.

### DETAILED DESCRIPTION OF THE INVENTION

The compositions and methods provided herein are well-suited for use in reducing off-target effects of viral vector-mediated therapies, including reducing an immune response to the viral vector itself and reducing an undesired immune response or toxicity associated with vector-mediated expression of the gene product. Pharmaceutical compositions are provided comprising neutralizing antibodies to a viral vector capsid (or envelope). In the examples herein, the compositions comprise anti-AAV neutralizing antibodies. The methods are particularly well suited for systemic pre-treatment (passive immunization) prior to delivery of viral vectors to the central nervous system or to the eye.

A method for preventing off-target effects of CNS-delivered recombinant AAV vectors is provided comprising the passive transfer of AAV Nabs. The examples below show that pretreatment with IVIG containing anti-AAV NAb significantly reduced vector transduction in the liver and other peripheral organs, but not in the CNS. Thus, pretreatment with anti-AAV NAb represents a useful method for preventing off-target effects as a result of non-CNS transduction following delivery of an AAV gene therapy to the CNS.

As used herein, the term “vector” refers to any molecule or moiety that transports, transduces, or otherwise acts as a carrier of a heterologous molecule such as the polynucleotides. A “viral vector” is a vector having a capsid or envelope protein from a viral source that is preferably rendered replication-defective for delivery to subjects and that comprises one or more exogenous polynucleotide regions encoding or comprising a molecule of interest, e.g., a transgene, a polynucleotide encoding a polypeptide or multi-polypeptide, polypeptide(s) encoding an immunoglobulin (e.g., an antibody), a modulatory nucleic acid, a nuclease for editing, among others. In certain embodiments, the viral vectors is produced recombinantly.

In certain embodiments, the methods and compositions provided are utilized in patients being treated with an adenovirus capsid protein (for treatment with a recombinant adenovirus), a herpes simplex virus (for treatment with a recombinant herpes simplex virus vector), or a lentivirus (for treatment with a recombinant lentivirus). Within each of these vector categories, neutralizing antibodies may be serologically specific, but within this specificity may be viruses having the same capsid source or a different capsid source that is serologically cross-reactive with the capsid. Different virus capsids within each of the virus types, AAV, adenovirus, HSV, or lentivirus, may be serologically distinct or serologically cross-reactive.

As used herein, a “neutralizing antibody” or “NAb” binds specifically to a viral capsid or envelope and interferes with the infectivity of the virus or a recombinant viral vector having the viral capsid or envelope, thus preventing the recombinant viral vector from delivering effective amounts of a gene product encoded by an expression cassette in its vector genome. Various methods for assessing neutralizing antibodies in a patient's sera may be utilized. The term method and assay are used interchangeably. As used herein, the terms “neutralization assay” and “serum virus neutralization assay” refer to serological tests to detect the presence of systemic antibodies that prevent infectivity of a virus. Such assays may also qualitatively or quantitatively discern the binding capacity (e.g., magnitude) or efficiency of the antibodies to neutralize a target. Immunological assays include enzyme immunoassay (EIA), radioimmunoassay (RIA), which uses radioactive isotopes, fluoroimmunoassay (FIA) which uses fluorescent materials, chemiluminescent immunoassay (CLIA) which uses chemiluminescent materials and counting immunoassay (CIA) which employs particle-counting techniques, other modified assays such as western blot, immunohistochemistry (IHC) and agglutination. One of the most common enzyme immunoassay's is enzyme-linked immunosorbent assay (ELISA). Example of suitable methods include those previously described, e.g., R Calcedo, et al. Journal Infectious Diseases. 2009, 199:381-290; GUO, et al., “Rapid AAV_Neutralizing Antibody Determination with a Cell-Binding Assay”, Molecular Therapy: Methods & Clinical Development Vol. 13 Jun. 2019, T. Ito et al, “A convenient enzyme-linked immunosorbent assay for rapid screening of anti-adeno-associated virus neutralizing antibodies”, Ann Clin Biochem 2009:46: 508-510: US 2018/0356394A2 (Voyager Therapeutics). Additionally, commercial kits exist (see, e.g., Athena Diagnostics, Invitrogen. ThermoFisher.com: Covance).

The neutralization ability of an antibody is usually measured via the expression of a reporter gene such as luciferase (Luc) or a green fluorescent protein (GFP). In order to determine and compare the activity of a neutralizing antibody, the antibody tested may display a neutralizing activity of 50% or more in one of the neutralization assays described herein. In some examples, neutralizing capacity is determined by measuring the activity of a reporter gene product (e.g., luciferase or GFP). The neutralizing capacity of an antibody to a specific viral vector may be at least 50%, 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

As used herein, the term “NAb titer” is a measurement of how much neutralizing antibody (e.g., anti-AAV Nab) is produced which neutralizes the physiologic effect of its targeted epitope (e.g., an AAV). Anti-AAV NAb titers may be measured as described in, e.g., Calcedo, R., et al., Worldwide Epidemiology of Neutralizing Antibodies to Adeno-Associated Viruses. Journal of Infectious Diseases, 2009, 199 (3): p. 381-390, which is incorporated by reference herein.

In certain embodiments, prior to anti-viral vector (e.g., anti-AAV) pretreatment, a patient for receipt of a viral vector (e.g., for gene therapy or gene editing) is screened to determine the neutralizing antibody titer of the patient to the capsid or envelope of the viral vector(s) to be delivered. In certain embodiment, a patient is determined to have no detectable anti-vector (e.g., anti-AAV antibodies) neutralizing antibodies prior to pretreatment (e.g., IVIG) and subsequent viral vector-mediated gene delivery to the CNS (or to the eye). In certain embodiments, the patient has a neutralizing antibody titer to the vector capsid or a serologically cross-reactive capsid which is less than a preselected value as determined in an in vitro assay: in such instance, the patient is pretreated with anti-viral vector antibodies that are neutralizing to the viral delivery vector. In certain embodiments, the patient is treated with the same anti-vector neutralizing antibodies for which they tested positive. In other embodiments, a patient tests positive for above a threshold amount of anti-vector neutralizing antibodies to a virus that is serologically distinct from the viral vector to be delivered. For example, where a patient sample is assessed as having an anti-AAV antibody titer to a selected capsid (e.g., AAV2 or AAV8) and the patient is to receive treatment with a Clade F capsid (e.g., AAV9, AAVhu68, AAVhu95 or AAVhu96), the patient may be passively immunized (e.g., pretreated) with an effective amount of anti-AAV Clade F neutralizing antibodies. Still other combinations may be selected.

In certain embodiments, an effective amount of anti-AAV antibodies is delivered to provide the patient with at least about 1:5 or greater anti-AAV neutralizing antibodies for the capsid found in the recombinant AAV vector, or a serologically cross-reactive antibody. In other embodiments, the patient is provided with an anti-AAV neutralizing titer greater than 1:10, greater than at least 1:20, at least about 1:50, at least about 1:100, at least about 1:500, at least about 1:1000, at least about 1:1280. In certain embodiments, the neutralizing antibody titer does not exceed about 1:2500. Similar titers may be selected for another type of vector to be delivered (e.g., adenovirus, etc). In certain embodiments, an effective amount is determined by the reduction or prevention of expression of the gene product delivered by the recombinant AAV to liver, heart, spleen, muscle, and/or another organ by comparison to a predetermined standard. In certain embodiments, an effective amount is determined by increased expression in target cell.

In certain embodiments, a composition comprising a human immunoglobulins directed against the selected viral vector (e.g., AAV, adenovirus, or lentivirus) contain anti-virus neutralizing antibodies from any suitable source. In certain embodiments, the antibodies are anti-AAV immunoglobulin constructs (e.g., polyclonal antibodies, monoclonal antibodies, affinity ligands).

The term “immunoglobulin” as used herein includes antibodies, functional fragments thereof, and immunoadhesins. Antibodies may exist in a variety of forms including, for example, polyclonal antibodies, monoclonal antibodies, camelid single domain antibodies, intracellular antibodies (“intrabodies”), recombinant antibodies, multispecific antibody, antibody fragments, such as, Fv, Fab, F(ab)₂, F(ab)₃, Fab′, Fab-SH, F(ab)₂, single chain variable fragment antibodies (scFv), tandem/bis-scFv, Fc, pFc′, scFvFc (or scFv-Fc), disulfide Fv (dsfv), bispecific antibodies (bc-scFv) such as BiTE antibodies; camelid antibodies, resurfaced antibodies, humanized antibodies, fully human antibodies, single-domain antibody (sdAb, also known as NANOBODY®), chimeric antibodies, chimeric antibodies comprising at least one human constant region, and the like. “Antibody fragment” refers to at least a portion of the variable region of an immunoglobulin that binds to its target, e.g., the cell-surface antigen or receptor.

In certain embodiments, the antibodies are humanized anti-AAV antibodies. Compositions may contain combinations of neutralizing antibodies or other immunoglobulin constructs (e.g., polyclonal and monoclonal antibodies, or combinations of polyclonal antibodies from different sources, or combinations or monoclonal antibodies (e.g., a cocktail)). Examples of suitable antibodies include, human anti-AAV antibodies (see, e.g., WO 2016/176212, and the sequences disclosed therein, providing anti-AAV antibodies capable of binding, e.g., AAV2, AAV3B, AAVrh10, AAV9, AAV8, and cross-reactive antibodies: WO 2021/257497 (anti-AAVrh74 antibodies): WO 00/26254; and WO 95/11977). Other sources of antibodies include anti-AAV antibodies isolated from non-human primates or other mammalian sources. Optionally these antibodies are “humanized”.

In certain embodiments, IG is obtained from pooled plasma from human patients having high neutralizing antibody levels against the selected target. In another example, PRIVIGEN® is selected as the immunoglobulin to be delivered intravenously (IVIG). Other commercially available IVIG products include those that are delivered IV (e.g., GAMMAGARD® (lyophilized; Baxter Healthcare)) or via another suitable route, e.g., subcutaneously (see, e.g., HIZENTRA®). However, these products may not always contain sufficient levels of anti-vector (e.g., anti-AAV) neutralizing antibodies and it may be necessary to fortify this product by admixing it with additional antibodies.

In other embodiments, the composition comprises pharmaceutically acceptable grades of AAV-specific affinity ligands [e.g., M. Mietzsch, et al., Characterization of AAV-Specific Affinity Ligands: Consequences for Vector Purification and Development Strategies, Molecular Therapy: Methods & Clinical Development Vol. 19 Dec. 2020, pp. 362-373, which is incorporated herein by reference] and/or peptide affinity reagents [see, e.g., Pulicherla, N., Asokan, A. Peptide affinity reagents for AAV capsid recognition and purification. Gene Ther 18, 1020-1024 (2011) and WO 2020/242988 (AAV affinity agent), which are incorporated herein by reference].

In certain embodiments, the IVIG compositions provided herein are obtained from commercial or other sources. Additionally or alternatively, the pharmaceutical compositions comprising a pharmaceutically acceptable suspension agent, diluent, carrier, and optional excipients and/or preservatives are formulated with one or more anti-viral vector neutralizing immunoglobulin constructs (e.g., an immunoglobulin source, polyclonal antibody, monoclonal antibody, affinity ligand, peptide affinity ligand, and/or combinations thereof) according to methods known to those of skill in the art. The description of suitable pharmaceutical components, e.g., buffered saline, surfactants, and the like, from the discussion of the viral vector is incorporated by reference herein.

An effective amount of anti-viral vector antibodies is delivered by any suitable route (e.g., intravenous, subcutaneous, intrahepatic, etc) to provide passive immunity to the patient. In certain embodiments, the passive immunization is short-lived, e.g., from days to weeks. In certain embodiments, the passive immunization is the same or exceeds the circulating half-life of the viral vector.

Suitable doses and dosing regimens of anti-viral vector neutralizing antibodies may be determined. For example, the doses and regimens for the commercially available products, e.g., PRIVIGEN®, GAMMAGARD®, or HIZENTRA®, may be obtained from their respective product literature. In some embodiments, a dose of anti-viral vector neutralizing antibodies is about 500 mg to about 2500 mg daily. However, other suitable doses may be selected. In certain embodiments, the patient may be treated with the anti-vector NAb composition at least two hours prior to treatment and on the days and/or weeks prior to treatment, e.g., one to seven days prior to administration of the viral vector, on the same day as the viral vector, and/or for a day, 3×/week for 1 to 2 weeks prior to viral vector delivery, weeks, or months.

In certain embodiments, following administration of anti-viral vector neutralizing antibodies via a suitable systemic route, the viral vector itself is administered to the CNS or to the eye. Routes of delivery for the viral vector are described below following the description of illustrative viral vectors.

In certain embodiments, the anti-AAV compositions and methods are used for treating off-target effects of gene therapy vectors delivered to the eye. Common routes of administration to the eye include intravitreal, intra-retinal, subretinal, or suprachoroidal administration.

### Expression Cassette

The regimens and methods provide include pre-treating a patient with neutralizing antibodies to a viral vector prior to administering the viral vector. The viral vectors comprise an expression cassette comprising a nucleic acid sequence encoding a gene product for expression in a target cell and regulatory sequences that direct expression of the gene product in a target cell when administered to a patient without neutralizing antibodies to the viral vector or when administered with according to regimens and methods provided herein.

As used herein, an “expression cassette” refers to a nucleic acid molecule comprising a biologically useful nucleic acid sequence (e.g., a gene cDNA encoding a protein, enzyme, or other useful gene product, mRNA, etc.) and regulatory sequences operably linked thereto that direct or modulate transcription, translation, and/or expression of the nucleic acid sequence and its gene product.

As used herein, “operably linked” sequences include both regulatory sequences that are contiguous or non-contiguous with the nucleic acid sequence and regulatory sequences that act in trans or cis to the nucleic acid sequence. Such regulatory sequences typically include, e.g., one or more of a promoter, an enhancer, an intron, a Kozak sequence, a polyadenylation sequence, and a TATA signal. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region (3′ UTR) comprising a polyadenylation site, among other elements. In certain embodiments, the regulatory sequences are operably linked to the nucleic acid sequence of a gene product, wherein the regulatory sequences are separated from nucleic acid sequence of a gene product by an intervening nucleic acid sequence, i.e., 5′-untranslated regions (5′UTR). In certain embodiments, the expression cassette comprises nucleic acid sequences of one or more gene products. In some embodiments, the expression cassette can be a monocistronic or a bicistronic expression cassette. In other embodiments, the term “transgene” refers to one or more DNA sequences from an exogenous source which are delivered to and/or expressed in a target cell.

Typically, such an expression cassette can be used for generating a vector genome for a viral vector and contains the coding sequence for a gene product described herein flanked by packaging signals of the viral genome and other expression control sequences such as those described herein. In certain embodiments, a vector genome contains two or more expression cassettes. Optionally, an expression cassette (and a vector genome) comprises one or more dorsal root ganglion (drg)-miRNA targeting sequences in the 3′ and/or 5′ UTR, e.g., to reduce drg-toxicity and/or axonopathy. See, e.g., WO 2020/132455, WO 2021/231579, and US Provisional Patent Application No. 63/279,561, filed Nov. 15, 2021, which are all incorporated by reference herein.

As used herein, a “vector genome” refers to the nucleic acid sequence packaged inside a parvovirus (e.g., AAV) capsid which forms a viral particle. Such a nucleic acid sequence contains AAV inverted terminal repeat (ITR) sequences. In the examples herein, a vector genome contains, at a minimum, from 5′ to 3′, an AAV 5′ ITR, coding sequence(s) (i.e., transgene(s)), and an AAV 3′ ITR. ITR sequences from AAV2, a different source AAV than the capsid, or other than full-length ITRs may be selected. In certain embodiments, the ITR sequences are from the same AAV source as the AAV which provides the rep function during production or a transcomplementing AAV. Further, other ITR sequences, e.g., self-complementary (scAAV) ITR sequences, may be used. Both single-stranded AAV and self-complementary (sc) AAV are encompassed with the recombinant AAV. The transgene is a nucleic acid coding sequence heterologous to the vector sequences and encodes a polypeptide, protein, functional RNA molecule (e.g., miRNA, miRNA inhibitor), or other gene product of interest. The nucleic acid coding sequence is operatively linked to regulatory components in a manner that permits transgene transcription, translation, and/or expression in a cell of a target tissue. Suitable components of a vector genome are discussed in more detail herein. In one example, a “vector genome” contains, at a minimum, from 5′ to 3′, a vector-specific sequence, a nucleic acid sequence encoding the gene product operably linked to regulatory control sequences (that direct expression of the gene product in a target cell), where the vector-specific sequence may be a terminal repeat sequence which specifically packages the vector genome into a viral vector capsid or envelope protein. For example, AAV ITR sequences are utilized for packaging a vector genome into an AAV capsid and certain other parvovirus capsids.

In certain embodiments, provided herein is an expression cassette comprising an engineered nucleic acid sequence encoding a nucleic acid sequence (transgene) encoding a desired gene product, and one or more regulatory sequences that direct expression of the gene product. In one embodiment, provided herein is an expression cassette comprising an engineered nucleic acid sequence as described herein encoding a functional gene product, and one or more regulatory sequences that direct expression of the engineered nucleic acid sequence and gene product.

In certain embodiments, the expression cassette contains any suitable transgene for delivery to a patient. Particularly suitable are expression cassettes that are to be delivered systemically via the viral vector. Examples of useful genes, coding sequences, and gene products are provided below in the section related to methods of use.

As used herein, the term “expression” or “gene expression” refers to the process by which information from a gene is used in the synthesis of a functional gene product. The gene product may be a protein, a peptide, or a nucleic acid polymer (such as an RNA, a DNA, or a PNA).

As used herein, the terms “regulatory sequence” or “expression control sequence” refer to nucleic acid sequences, such as initiator sequences, enhancer sequences, and promoter sequences, which induce, repress, or otherwise control the transcription of protein encoding nucleic acid sequences to which they are operably linked.

The term “exogenous” as used to describe a nucleic acid sequence or protein means that the nucleic acid or protein does not naturally occur in the position in which it exists in a chromosome, or host cell. An “exogenous nucleic acid sequence” also refers to a sequence derived from and inserted into the same host cell or subject, but which is present in a non-natural state, e.g., a different copy number, or under the control of different regulatory elements.

The term “heterologous” as used to describe a nucleic acid sequence or protein means that the nucleic acid or protein was derived from a different organism or a different species of the same organism than the host cell or subject in which it is expressed. The term “heterologous” when used with reference to a protein or a nucleic acid in a plasmid, expression cassette, or vector (e.g., rAAV), indicates that the protein or the nucleic acid is present with another sequence or subsequence which with which the protein or nucleic acid in question is not found in the same relationship to each other in nature.

In certain embodiments, a constitutive promoter is be selected. In one embodiment, the promoter is human cytomegalovirus (CMV) or a chicken β-actin promoter. A variety of chicken beta-actin promoters have been described alone, or in combination with various enhancer elements (e.g., CB7 is a chicken beta-actin promoter with cytomegalovirus enhancer elements: a CAG promoter, which includes the promoter, the first exon and first intron of chicken beta actin, and the splice acceptor of the rabbit beta-globin gene: a CBh promoter, S J Gray et al, Hu Gene Ther, 2011 September; 22 (9): 1143-1153). Alternatively, other constitutive promoters may be selected.

In certain embodiments, a tissue-specific promoter is selected for the expression cassette and vector genome of the viral vector. For example, for CNS-mediated delivery, a promoter may be selected from promoters useful in the central nervous system (e.g., neurons or subsets thereof). In certain embodiments, the promoter is a neuron-specific promoter. Examples of neuron-specific promoters include, e.g., an elongation factor 1 alpha (EF1 alpha) promoter (see, e.g., Kim D W et al, Use of the human elongation factor 1 alpha promoter as a versatile and efficient expression system. Gene. 1990 Jul. 16; 91 (2): 217-23), a Synapsin 1 promoter (see, e.g., Kügler S et al, Human synapsin 1 gene promoter confers highly neuron-specific long-term transgene expression from an adenoviral vector in the adult rat brain depending on the transduced area. Gene Ther. 2003 February; 10 (4): 337-47), a shorted synapsin promoter, a neuron-specific enolase (NSE) promoter (see, e.g., Kim J et al, Involvement of cholesterol-rich lipid rafts in interleukin-6-induced neuroendocrine differentiation of LNCaP prostate cancer cells. Endocrinology. 2004 February; 145 (2): 613-9. Epub 2003 Oct. 16), or a CB6 promoter (see, e.g., Large-Scale Production of Adeno-Associated Viral Vector Serotype-9 Carrying the Human Survival Motor Neuron Gene, Mol Biotechnol., 2016 January; 58 (1): 30-6).

In certain embodiments, for ocular (eye) vector delivery, a promoter is chosen from a promoter specific for expression of the transgene in retinal pigment epithelial cells. In certain embodiments, the promoter is specific for expression of the transgene in photoreceptor cells. In certain embodiments, the promoter is specific for expression in rods and cones. In certain embodiments, the promoter is specific for expression in the rods. In certain embodiment, the promoter is specific for expression in the cones. In certain embodiments, the photoreceptor-specific promoter is a human rhodopsin kinase promoter. In certain embodiments, the promoter is the native hVMD2 promoter or a modified version thereof. See Guziewicz et al., PLOS One. 2013 Oct. 15; 8 (10): e75666, which is incorporated herein by reference. In certain embodiments, the promoter is a human rhodopsin promoter. See, e.g, Nathans and Hogness, Isolation and nucleotide sequence of the gene encoding human rhodopsin. PNAS, 81:4851-5 (August 1984), which is incorporated herein by reference in its entirety. In certain embodiments, the promoter is a portion or fragment of the human rhodopsin promoter. In certain embodiments, the promoter is a variant of the human rhodopsin promoter. Other exemplary promoters include the human G-protein-coupled receptor protein kinase 1 (GRK1) promoter (Genbank Accession number AY327580). In certain embodiments, the promoter is a 292 nt fragment (positions 1793-2087) of the GRK1 promoter (See, Beltran et al, Gene Therapy 2010 17:1162-74, which is hereby incorporated by reference in its entirety). In certain embodiments, the promoter is the human interphotoreceptor retinoid-binding protein proximal (IRBP) promoter. In certain embodiments, the promoter is a 235 nt fragment of the hIRBP promoter. In certain embodiments, the promoter is the RPGR proximal promoter (Shu et al, IOVS, May 2102, which is incorporated by reference in its entirety). Other promoters useful in the invention include, without limitation, the rod opsin promoter, the red-green opsin promoter, the blue opsin promoter, the cGMP-β-phosphodiesterase promoter (Qgueta et al. IOVS, Invest Ophthalmol Vis Sci. 2000 December; 41 (13): 4059-63), the mouse opsin promoter (Beltran et al 2010 cited above), the rhodopsin promoter (Mussolino et al, Gene Ther, July 2011, 18 (7): 637-45): the alpha-subunit of cone transducin (Morrissey et al, BMC Dev, Biol, January 2011, 11:3); beta phosphodiesterase (PDE) promoter; the retinitis pigmentosa (RP1) promoter (Nicord et al, J. Gene Med, December 2007, 9 (12): 1015-23): the NXNL2/NXNL1 promoter (Lambard et al, PLOS One, October 2010, 5 (10): e13025), the RPE65 promoter: the retinal degeneration slow/peripherin 2 (Rds/perph2) promoter (Cai et al, Exp Eye Res. 2010 August; 91 (2): 186-94); and the VMD2 promoter (Kachi et al, Human Gene Therapy, 2009 (20:31-9)). Each of these documents is incorporated by reference herein in its entirety. In another embodiment, the promoter is selected from human EF1α promoter, rhodopsin promoter, rhodopsin kinase, interphotoreceptor binding protein (IRBP), cone opsin promoters (red-green, blue), cone opsin upstream sequences containing the red-green cone locus control region, cone transducing, and transcription factor promoters (neural retina leucine zipper (Nrl) and photoreceptor-specific nuclear receptor Nr2e3, bZIP).

Alternatively, a regulatable promoter may be selected. See, e.g., WO 2017/106244, which describes different regulatable expression systems and the rapamycin/rapalog inducible systems, and WO2007/126798, U.S. Pat. No. 6,506,379, and WO 2011/126808B2, each of which is incorporated by reference herein. In certain embodiments, the regulatory sequence further comprises an enhancer. In certain embodiments, the regulatory sequence comprises one enhancer. In certain embodiments, the regulatory sequence contains two or more expression enhancers. The enhancers may be the same or different. For example, an enhancer may include an alpha mic/bik enhancer or a CMV enhancer. The enhancers may be present in two copies which are located adjacent to one another. Alternatively, the dual copies of the enhancer may be separated by one or more sequences.

In certain embodiments, the regulatory sequences further comprise an intron. In certain embodiments, the intron is a chicken beta-actin intron. Other suitable introns include those known in the art including a human β-globulin intron, and/or a commercially available Promega® intron, and those described in WO 2011/126808.

In certain embodiments, the regulatory sequences further comprise a polyadenylation signal (polyA). In certain embodiments, the polyA is a rabbit globin poly A. See, e.g., WO 2014/151341. Alternatively, another polyA, e.g., a human growth hormone (hGH) polyadenylation sequence, an SV40 polyA, a thymidine kinase (TK) or a synthetic polyA may be included in an expression cassette.

It should be understood that the compositions in the expression cassette described herein are intended to be applied to other compositions, regiments, aspects, embodiments, and methods described across the specification.

### Vector

In certain embodiments, provided herein is a vector comprising an engineered nucleic acid sequence encoding a functional human gene product and one or more regulatory sequences that direct expression of the transgene in a target cell in the central nervous system, or a subset of cells of the central nervous system, or in the eye, or a subset of cells in the eye. In certain embodiments, combinations of these vectors are used.

In certain embodiments, the vector described herein is a “replication-defective virus” or a “viral vector” which refers to a synthetic or artificial viral particle in which an expression cassette containing a nucleic acid sequence encoding a functional gene product(s) is packaged in a viral capsid or envelope, where any viral genomic sequences also packaged within the viral capsid or envelope are replication-deficient: i.e., they cannot generate progeny virions but retain the ability to infect target cells. In certain embodiments, the vector genome of the vector does not include genes encoding the enzymes required to replicate (the genome can be engineered to be “gutless”-containing only the nucleic acid sequence encoding the gene product flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

Suitable viral vectors include any suitable delivery vector, such as, e.g., a recombinant adenovirus, a recombinant lentivirus, a recombinant bocavirus, a recombinant AAV, or another recombinant parvovirus (e.g., bocavirus or hybrid AAV/bocavirus), a retroviral vector, adenoviral vector, poxviral vector (e.g., vaccinia viral vector, such as modified vaccinia ankara (MVA)), or alphaviral vector). In certain embodiments, the viral vector is a recombinant AAV for delivery of a gene product to a patient in need thereof.

As used herein, a host cell may be a packaging cell line is used for production of a vector (e.g., a recombinant AAV). The cell line may be a prokaryotic or eukaryotic cell (e.g., human, insect, or yeast) that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection, or protoplast fusion. Examples of cells include, but are not limited to an isolated cell, a cell culture, an Escherichia coli cell, a yeast cell, a human cell, a non-human cell, a mammalian cell, a non-mammalian cell, an insect cell, an HEK-293 cell, a liver cell, a kidney cell, a cell of the central nervous system, a neuron, a glial cell, or a stem cell.

As used herein, the term “target cell” refers to any target cell in which expression of the functional gene product is desired. In certain embodiments, the term “target cell” is intended to reference the cells of the subject being treated. Examples of target cells include, but are not limited to, cells in the eye and/or one or more subsets of cells of the eye (e.g., photoreceptors, retinal cells, retinal pigmented epithelial cells, rods, cones, pigment epithelial cells, Müller glia, retinal ganglion cells, and/or others). Other suitable target cells are the CNS and/or in subsets of CNS cells (including brain, astrocytes, neurons, ependymal cells, and cells of the choroid plexus). In certain embodiments, delivery to the CNS excludes injection/delivery to the eye.

In certain embodiments, IVIG and/or other anti-AAV compositions are delivered systemically to prevent cardiotoxic effects of CNS-mediated delivery of AAV.trastuzumab. See. e.g., WO 2015/164723 (anti-Her antibody expressed from AAV) and WO 2018/160582, both of which are incorporated herein by reference.

For expression from a viral vector (e.g., AAV), nucleic acid constructs which encode immunoglobulins useful in treatment of one or more neurodegenerative disorders may be engineered or selected for delivery via an AAV composition of the invention. Such disorders may include, without limitation, transmissible spongiform encephalopathies (e.g., Creutzfeld-Jacob disease), Parkinson's disease, amyotropic lateral sclerosis (ALS), multiple sclerosis, Alzheimer's Disease, Huntington disease, Canavan's disease (e.g., associated with mutations in the aspartoacylase (ASPA) gene), traumatic brain injury, spinal cord injury (ATI335, anti-nogo1 by Novartis), migraine (ALD403 by Alder Biopharmaceuticals; LY2951742 by Eli; RN307 by Labrys Biologics), lysosomal storage diseases, stroke, and infectious disease affecting the central nervous system.

Still other nucleic acids may encode an immunoglobulin which is directed to leucine rich repeat and immunoglobulin-like domain-containing protein 1 (LINGO-1), which is a functional component of the Nogo receptor and which is associated with essential tremors in patients which multiple sclerosis, Parkinson's Disease or essential tremor. One such commercially available antibody is ocrelizumab (Biogen, BIIB033). See, e.g., U.S. Pat. No. 8,425,910.

In certain embodiments, the nucleic acid construct encodes immunoglobulin constructs useful for patients with ALS. Examples of suitable antibodies include antibodies against the ALS enzyme superoxide dismutase 1 (SOD1) and variants thereof (e.g., ALS variant G93A, C4F6 SOD1 antibody); MS785, which directed to Derlin-1-binding region); antibodies against neurite outgrowth inhibitor (NOGO-A or Reticulon 4), e.g., GSK1223249, ozanezumab (humanized, GSK, also described as useful for multiple sclerosis).

In certain embodiments, the nucleic acid sequences are be designed or selected which encode immunoglobulins useful in patients having Alzheimer's Disease. Such antibody constructs include, e.g., adumanucab (Biogen), Bapineuzumab (Elan: a humanised mAb directed at the amino terminus of Aβ); Solanezumab Eli Lilly, a humanized mAb against the central part of soluble Aβ); Gantenerumab (Chugai and Hoffmann-La Roche, is a full human mAb directed against both the amino terminus and central portions of Aβ); Crenezumab (Genentech, a humanized mAb that acts on monomeric and conformational epitopes, including oligomeric and protofibrillar forms of Aβ; BAN2401 (Esai Co., Ltd, a humanized immunoglobulin G1 (IgG1) mAb that selectively binds to AB protofibrils and is thought to either enhance clearance of AB protofibrils and/or to neutralize their toxic effects on neurons in the brain); GSK 933776 (a humanised IgG1 monoclonal antibody directed against the amino terminus of Aβ); AAB-001. AAB-002, AAB-003 (Fc-engineered bapineuzumab); SAR228810 (a humanized mAb directed against protofibrils and low molecular weight Aβ): BIIB037/BART (a full human IgG1 against insoluble fibrillar human Aβ, Biogen Idec), an anti-Aβ antibody such m266, tg2576 (relative specificity for AB oligomers) [Brody and Holtzman, Annu Rev Neurosci, 2008; 31: 175-193]. Other antibodies may be targeted to beta-amyloid proteins, Aβ, beta secretase and/or the tau protein.

Nucleic acids encoding other immunoglobulin constructs for treatment of patients with Parkinson's disease may be engineered or designed to express constructs, including, e.g., leucine-rich repeat kinase 2, dardarin (LRRK2) antibodies, anti-synuclein and alpha-synuclein antibodies and DJ-1 (PARK7) antibodies. Other antibodies may include, PRX002 (Prothena and Roche) Parkinson's disease and related synucleinopathies. These antibodies, particularly anti-synuclein antibodies may also be useful in treatment of one or more lysosomal storage disease.

One may engineer or select nucleic acid constructs encoding an immunoglobulin construct for treating multiple sclerosis. Such immunoglobulins may include or be derived from antibodies such as natalizumab (a humanized anti-a4-ingrin, iNATA, Tysabri, Biogen Idec and Elan Pharmaceuticals), which was approved in 2006, alemtuzumab (Campath-1H, a humanized anti-CD52), rituximab (rituzin, a chimeric anti-CD20), daclizumab (Zenepax, a humanized anti-CD25), ocrelizumab (humanized, anti-CD20, Roche), ustekinumab (CNTO-1275, a human anti-IL 12 p40+IL23p40): anti-LINGO-1, and ch5D12 (a chimeric anti-CD40), and rHIgM22 (a remyelinated monoclonal antibody: Acorda and the Mayo Foundation for Medical Education and Research). Still other anti-a4-integrin antibodies, anti-CD20 antibodies, anti-CD52 antibodies, anti-IL17, anti-CD19, anti-SEMA4D, and anti-CD40 antibodies may be delivered via the AAV vectors as described herein.

AAV-mediated delivery of antibodies against various infections of the central nervous system is also contemplated. Such infectious diseases include fungal diseases such as cryptoccocal meningitis, brain abscess, spinal epidural infection caused by, e.g., Cryptococcus neoformans, Coccidioides immitis, order Mucorales, Aspergillus spp, and Candida spp; protozoal, such as toxoplasmosis, malaria, and primary amoebic meningoencepthalitis, caused by agents such as, e.g., Toxoplasma gondii, Taenia solium, Plasmodium falciparus, Spirometra mansonoides (sparaganoisis), Echinococcus spp (causing neuro hydatosis), and cerebral amoebiasis; bacterial, such as, e.g., tuberculosis, leprosy, neurosyphilis, bacterial meningitis, lyme disease (Borrelia burgdorferi). Rocky Mountain spotted fever (Rickettsia rickettsia). CNS nocardiosis (Nocardia spp), CNS tuberculosis (Mycobacterium tuberculosis), CNS listeriosis (Listeria monocytogenes), brain abscess, and neuroborreliosis: viral infections, such as, e.g., viral meningitis, Eastern equine encephalitis (EEE), St Louis encepthalitis, West Nile virus and/or encephalitis, rabies, California encephalitis virus, La Crosse encephalitis, measles encephalitis, poliomyelitis, which may be caused by, e.g., herpes family viruses (HSV), HSV-1, HSV-2 (neonatal herpes simplex encephalitis), varicella zoster virus (VZV), Bickerstaff encephalitis, Epstein-Barr virus (EBV), cytomegalovirus (CMV, such as TCN-202 is in development by Theraclone Sciences), human herpesvirus 6 (HHV-6), B virus (herpesvirus simiae), Flavivirus encephalitis, Japanese encephalitis, Murray valley fever. JC virus (progressive multifocal leukoencephalopathy), Nipah Virus (NiV), measles (subacute sclerosing panencephalitis); and other infections, such as, e.g., subactuate sclerosing panencephalitis, progressive multifocal leukoencephalopathy; human immunodeficiency virus (acquired immunodeficiency syndrome (AIDS)); Streptococcus pyogenes and other β-hemolytic Streptococcus (e.g., Pediatric Autoimmune Neuropsychiatric Disorders Associated with Streptococcal Infection, PANDAS) and/or Syndenham's chorea, and Guillain-Barre syndrome, and prions.

Examples of suitable antibody constructs include those described, e.g., in WO 2007/012924A2, Jan. 29, 2015, which is incorporated by reference herein.

In certain embodiments, nucleic acid sequences encode anti-prion immunoglobulin constructs. Such immunoglobulins may be directed against major prion protein (PrP, for prion protein or protease-resistant protein, also known as CD230 (cluster of differentiation 230). The amino acid sequence of PrP is provided, e.g., http://www.ncbi.nlm.nih.gov/protein/NP_000302, incorporated by reference herein. The protein can exist in multiple isoforms, the normal PrPC, the disease-causing PrPSc, and an isoform located in mitochondria. The misfolded version PrPSc is associated with a variety of cognitive disorders and neurodegenerative diseases such as Creutzfeldt-Jakob disease, bovine spongiform encephalopathy, Gerstmann-Sträussler-Scheinker syndrome, fatal familial insomnia, and kuru.

In certain embodiments, the recombinant AAV is used in a gene editing system, which system may involve one recombinant AAV or co-administration of multiple recombinant AAV stocks, optionally in combination with another moiety (e.g., LNP). For example, the recombinant AAV may be engineered to deliver a nuclease or delivered in combination with another moiety which delivers SpCas9, SaCas9, ARCUS, Cpf1 (also known as Cas12a), CjCas9, and other suitable gene editing constructs. Additionally or alternatively, an rAAV may be desired for use in gene suppression therapy, i.e., expression of one or more native genes is interrupted or suppressed at transcriptional or translational levels. This can be accomplished using short hairpin RNA (shRNA) or other techniques well known in the art. See, e.g., Sun et al, Int J Cancer. 2010 Feb. 1; 126 (3): 764-74 and O'Reilly M, et al. Am J Hum Genet. 2007 July; 81 (1): 127-35, which are incorporated herein by reference. In this embodiment, the transgene may be readily selected by one of skill in the art based upon the gene which is desired to be silenced. In certain embodiments, the recombinant AAV has an expression cassette comprising at least one miRNA target sequences. In certain embodiments, the recombinant AAV comprises the at least one miRNA targeting sequences, wherein the miRNA is a dorsal root ganglion (drg)-miRNA targeting sequences, e.g., to reduce drg toxicity and/or axonopathy, such as are described above.

In certain embodiments, the anti-AAV NAb compositions (e.g., IVIG) in pre-treatments are used in combination with recombinant AAV vectors to be delivered directly to the eye. Examples of suitable genes for delivery to the eye include, e.g., sFLt-1, endostatin, angiostatin, anti-VEGF, TIMP3, PEDF, for treatment of neovascularization, macular degeneration (e.g., age-related macular degeneration (AMD), wet AMD), Prph2, Rho, BPDE, Bcl2, PEGF, FGF-2, epo, CNTF, Mertk, e.g., for treatment of retinitis pigmentosa: GUCY2D, AIPL1, PRGRIP, RPE65, for treatment of Leber congenital amaurosis: GNAT2 for treatment of achromatopsia: ABCA4, for treatment of Stargardt disease: Rs1 for treatment of retinoschisis; BDNF, CNTF, or GDNF, for treatment of glaucoma; IL10, IL-1Ra, for treatment of uveitis; IFN-β or TK for treatment of retinoblastoma; L-opsin for treatment of red-green color blindness; and/or GABP for treatment of corneal neovascularization.

It should be understood that the compositions in the vector described herein are intended to be applied to other compositions, regimens, aspects, embodiments, and methods described across the Specification.

### Recombinant AAV Vectors

In one aspect, provided herein is a recombinant AAV comprising an AAV capsid and a vector genome packaged therein. In certain embodiments, the vector genome comprises an AAV 5′ inverted terminal repeat (ITR) sequence, an engineered nucleic acid sequence encoding a gene product as described herein, one or more regulatory sequences that direct expression of the gene product in a target cell, and an AAV 3′ ITR sequence. The vector genome comprises an AAV 5′ ITR sequence, an engineered nucleic acid sequence encoding a gene product as described herein, a regulatory sequence which direct expression of the gene product a target cell, and an AAV 3′ ITR sequence. In certain embodiments, the regulatory sequences comprise a tissue-specific promoter (e.g., muscle- or liver-specific promoter). In certain embodiments, the regulatory sequences comprise an enhancer. In certain embodiment, the regulatory sequences further comprise an intron. In certain embodiment, the regulatory sequences further comprises a poly A. In certain embodiments, the AAV capsid is an AAV1 capsid. In certain embodiments, the AAV capsid is well-suited for delivery to the eye (e.g., an AAV2 capsid or AAV8 capsid). In certain embodiments, the AAV capsid is well-suited for delivery to the central nervous system (e.g, an AAV9 capsid, an AAVhu68 capsid, an AAVhu95 capsid, an AAVhu96 capsid, an AAV1 capsid, an AAVrh91 capsid). In certain embodiments, the AAV capsid is an AAVhu68 capsid. In certain embodiments, the AAV capsid is an AAVrh91 capsid.

In certain embodiments, the one or more regulatory sequences are as described above. In certain embodiments, the vector genome comprises an AAV 5′ ITR sequence, an expression cassette as described herein, and an AAV 3′ ITR sequence. The ITR sequences are the genetic elements responsible for the replication and packaging of the genome during vector production and are the only viral cis elements required to generate recombinant AAV vector. In certain embodiment, the ITR sequences are from an AAV different than that supplying a capsid. In a preferred embodiment, the ITR sequences from AAV2, or the deleted version thereof (ΔITR), which may be used for convenience and to accelerate regulatory approval. However, ITRs from other AAV sources may be selected. Where the source of the ITRs is from AAV2 and the AAV capsid is from another AAV source, the resulting vector may be termed “pseudotyped.” Typically, AAV vector genome comprises an AAV 5′ ITR sequence, the nucleic acid sequences encoding the gene product(s) and any regulatory sequences, and an AAV 3′ ITR sequence. However, other configurations of these elements may be suitable. In certain embodiments, a self-complementary AAV is provided. A shortened version of the 5′ ITR, termed ΔITR, has been described in which the D-sequence and terminal resolution site (trs) are deleted. In certain embodiments, the vector genome includes a shortened AAV2 ITR sequence of 130 base pairs, wherein the external “a” elements is deleted. The shortened ITR is reverted back to the wild type length of 145 base pairs during vector DNA amplification using the internal A element as a template. In other embodiments, the full-length AAV 5′ and 3′ ITR sequences are used.

The term “AAV” as used herein refers to naturally occurring adeno-associated viruses, adeno-associated viruses available to one of skill in the art and/or in light of the composition(s) and method(s) described herein, as well as artificial AAVs. An adeno-associated virus (AAV) viral vector is an AAV Dnase-resistant particle having an AAV protein capsid into which is packaged expression cassette flanked by AAV inverted terminal repeat sequences (ITRs) for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral vectors as identified above. In one embodiment, the AAV capsid is an AAV9 capsid or variant thereof. In certain embodiments, the capsid protein is designated by a number or a combination of numbers and letters following the term “AAV” in the name of the rAAV vector. Unless otherwise specified, the AAV capsid, ITRs, and other selected AAV components described herein, may be readily selected from among any AAV, including, without limitation, the AAVs identified as AAV1, AAV2, AAV3, AAV4, AAV5, AAV6, AAV7, AAV8, AAV9, AAVrh10, AAVhu37, AAVrh32.33, AAV8 bp, AAV7M8 and AAVAnc80, AAV1, AAV2, AAV3, AAV4, AAV5, AAV6, AAV7, AAV8, AAV9.47, AAV9 (hu14), AAV10, AAV11, AAV12, AAVrh8, AAVrh74, AAV-DJ8, AAV-DJ, AAVhu68, without limitation, See, e.g., WO2019/168961 and WO 2019/169004 (AAV Vectors; Deamidation); WO 2019/169004 (novel AAV capsids); US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449, 7,282,199 (AAV8), WO 2005/033321, U.S. Pat. No. 7,906,111 (AAV9), WO 2006/110689, WO 2003/042397 (rh.10), and WO 2005/033321, which are incorporated herein by reference. Other suitable AAVs may include, without limitation, AAVrh90, AAVrh91, AAVrh92, AAVrh93, AAVrh91.93. See, e.g., WO 2020/223232 A1 (AAV rh.90), WO 2020/223231 A1 (AAVrh.91), WO 2020/223236 A1 (AAVrh.92, AAVrh.93, AAV rh.91.93), U.S. Provisional Application No. 63/251,599, filed Oct. 2, 2021 (AAVhu95), and U.S. Provisional Application No. 63/251,599, filed Oct. 2, 2021 (AAVhu96), or AAV9 variants (AAV9 variants for brain endothelial cells, PCT/US21/61312, filed Dec. 1, 2021), all of which are incorporated herein by reference in its entirety. Other suitable AAVs include AAV3B variants described in WO2021/080991, including AAV3B.AR2.01, AAV3B.AR2.02, AAV3B.AR2.03, AAV3B.AR2.04, AAV3B.AR2.05, AAV3B.AR2.06, AAV3B.AR2.07, AAV3B.AR2.08. AAV3B.AR2.10. AAV3B.AR2.11. AAV3B.AR2.12. AAV3B.AR2.13. AAV3B.AR2.14, AAV3B.AR2.15, AAV3B.AR2.16, or AAV3B.AR2.17, which is incorporated herein by reference. These cited documents also describe other AAVs which may be selected for generating recombinant AAV vectors and are incorporated herein by reference. Among the AAVs isolated or engineered from human or non-human primates (NHPs) and well-characterized, human AAV2 is the first AAV that was developed as a gene transfer vector: it has been widely used for efficient gene transfer experiments in different target tissues and animal models.

As used herein, relating to AAV, the term “variant” means any AAV sequence which is derived from a known AAV sequence, including those with a conservative amino acid replacement, and those sharing at least 70%, at least 75%, at least 80%, at least 85%, at least 90%, at least 95%, at least 97%, at least 99% or greater sequence identity over the amino acid or nucleic acid sequence.

In certain embodiments, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In one embodiment, the AAV capsid shares at least 95% identity with an AAV capsid. When determining the percent identity of an AAV capsid, the comparison may be made over any of the variable proteins (e.g., vp1, vp2, or vp3).

The ITR sequences or other AAV components may be readily isolated or engineered using techniques available to those of skill in the art from an AAV. Such AAV may be isolated, engineered, or obtained from academic, commercial, or public sources (e.g., the American Type Culture Collection, Manassas, VA). Alternatively, the AAV sequences may be engineered through synthetic or other suitable means by reference to published sequences such as are available in the literature or in databases such as, e.g., GenBank, PubMed, or the like. AAV viruses may be engineered by conventional molecular biology techniques, making it possible to optimize these particles for cell specific delivery of nucleic acid sequences, for minimizing immunogenicity, for tuning stability and particle lifetime, for efficient degradation, for accurate delivery to the nucleus, etc.

As used herein, the terms “rAAV”, “rAAV vector,” “rAAV particle,” or “recombinant AAV vector” are used interchangeably to mean, without limitation, an AAV comprising a capsid protein and a vector genome packaged therein, wherein the vector genome comprises a nucleic acid heterologous to the AAV. In certain embodiments, the capsid protein is a non-naturally occurring capsid. Such an artificial capsid may be generated by any suitable technique, using a selected AAV sequence (e.g., a fragment of a vp1 capsid protein) in combination with heterologous sequences which may be obtained from a different selected AAV, non-contiguous portions of the same AAV, from a non-AAV viral source, or from a non-viral source. An artificial AAV may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the invention. In one embodiment, AAV2/5 and AAV2/8 are exemplary pseudotyped vectors. The selected genetic element may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

Methods of generating a capsid, coding sequences therefore, and methods for production of recombinant AAV viral vectors have been described. See, e.g., Gao, et al, Proc. Natl. Acad. Sci. U.S.A. 100 (10), 6081-6086 (2003) and US 2013/0045186A1.

In certain embodiments, the recombinant AAV as described herein is a self-complementary AAV. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535, 7,125,717, and 7,456,683, each of which is incorporated herein by reference in its entirety.

In certain embodiments, the recombinant AAV described herein is nuclease-resistant. Such nuclease may be a single nuclease, or mixtures of nucleases, and may be endonucleases or exonucleases. A nuclease-resistant rAAV indicates that the AAV capsid has fully assembled and protects these packaged genomic sequences from degradation (digestion) during nuclease incubation steps designed to remove contaminating nucleic acids which may be present from the production process. In many instances, the rAAV described herein is DNase resistant.

The recombinant AAV described herein may be generated using techniques which are known. See, e.g., WO 2003/042397: WO 2005/033321, WO 2006/110689: U.S. Pat. No. 7,588,772 B2. Such methods involve culturing a host cell which contains a nucleic acid sequence encoding an AAV capsid: a functional rep gene: an expression cassette as described herein flanked by AAV inverted terminal repeats (ITRs); and sufficient helper functions to permit packaging of the expression cassette into the AAV capsid protein. Also provided herein is the host cell which contains a nucleic acid sequence encoding an AAV capsid: a functional rep gene: a vector genome as described; and sufficient helper functions to permit packaging of the vector genome into the AAV capsid protein. In one embodiment, the host cell is a HEK 293 cell. These methods are described in more detail in WO2017160360 A2, which is incorporated by reference herein.

Other methods of producing recombinant AAV vectors available to one of skill in the art may be utilized. Suitable methods may include without limitation, baculovirus expression system or production via yeast. See, e.g., Robert M. Kotin. Large-scale recombinant adeno-associated virus production. Hum Mol Genet. 2011 Apr. 15; 20 (R1): R2-R6; Aucoin M G et al., Production of adeno-associated viral vectors in insect cells using triple infection: optimization of baculovirus concentration ratios. Biotechnol Bioeng. 2006 Dec. 20; 95 (6): 1081-92; Sami S. Thakur, Production of Recombinant Adeno-associated viral vectors in yeast. Thesis presented to the Graduate School of the University of Florida, 2012; Kondratov O et al. Direct Head-to-Head Evaluation of Recombinant Adeno-associated Viral Vectors Manufactured in Human versus Insect Cells, Mol Ther. 2017 Aug. 10. pii: S1525-0016 (17) 30362-3: Mietzsch M et al, OneBac 2.0: Sf9 Cell Lines for Production of AAV1, AAV2, and AAV8 Vectors with Minimal Encapsidation of Foreign DNA. Hum Gene Ther Methods. 2017 February; 28 (1): 15-22; Li L et al. Production and characterization of novel recombinant adeno-associated virus replicative-form genomes: a eukaryotic source of DNA for gene transfer. pLoS One. 2013 Aug. 1; 8 (8): e69879; Galibert L et al, Latest developments in the large-scale production of adeno-associated virus vectors in insect cells toward the treatment of neuromuscular diseases. J Invertebr Pathol. 2011 July; 107 Suppl: S80-93; and Kotin R M, Large-scale recombinant adeno-associated virus production. Hum Mol Genet. 2011 Apr. 15; 20 (R1).

In certain embodiments, a two-step affinity chromatography purification at high salt concentration followed by anion exchange resin chromatography is used to purify the vector drug product and to remove empty capsids. These methods are described in more detail in WO 2017/160360 entitled “Scalable Purification Method for AAV9”, which is incorporated by reference herein. In brief, the method for separating recombinant AAV9 particles having packaged genomic sequences from genome-deficient AAV9 intermediates involves subjecting a suspension comprising recombinant AAV9 viral particles and AAV9 capsid intermediates to fast performance liquid chromatography, wherein the AAV9 viral particles and AAV9 intermediates are bound to a strong anion exchange resin equilibrated at a pH of 10.2, and subjected to a salt gradient while monitoring eluate for ultraviolet absorbance at about 260 and about 280. Although less optimal for rAAV9, the pH may be in the range of about 10.0 to 10.4. In this method, the AAV9 full capsids are collected from a fraction which is eluted when the ratio of A260/A280 reaches an inflection point. In one example, for the Affinity Chromatography step, the diafiltered product may be applied to a Capture Select™ Poros-AAV2/9 affinity resin (Life Technologies) that efficiently captures the AAV2/9 serotype. Under these ionic conditions, a significant percentage of residual cellular DNA and proteins flow through the column, while AAV particles are efficiently captured.

Conventional methods for characterization or quantification of rAAV are available to one of skill in the art. To calculate empty and full particle content, VP3 band volumes for a selected sample (e.g., in examples herein an iodixanol gradient-purified preparation where #of GC=#of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL−GC/mL gives empty pt/mL. Empty pt/mL divided by pt/mL and ×100 gives the percentage of empty particles. Generally, methods for assaying for empty capsids and AAV vector particles with packaged genomes have been known in the art. See, e.g., Grimm et al., Gene Therapy (1999) 6:1322-1330; Sommer et al., Molec Ther (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Viral. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e., SYPRO stain. In one embodiment, the concentration of AAV vector genomes (vg) in column fractions can be measured by quantitative real time PCR (Q-PCR). Samples are diluted and digested with dNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad-spectrum serine protease, e.g., proteinase K (such as is commercially available from Qiagen). More particularly, the optimized qPCR genome titer assay is similar to a standard assay, except that after the dNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000-fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25 (2): 115-25.

Methods for determining the ratio among vp1, vp2 and vp3 of capsid protein are also available. See, e.g., Vamseedhar Rayaprolu et al, Comparative Analysis of Adeno-Associated Virus Capsid Stability and Dynamics, J Virol. 2013 December; 87 (24): 13150-13160; Buller R M, Rose J A. 1978. Characterization of adenovirus-associated virus-induced polypeptides in KB cells. J. Virol. 25:331-338; and Rose J A, Maizel J V, Inman J K, Shatkin A J. 1971. Structural proteins of adenovirus-associated viruses. J. Virol. 8:766-770.

As used herein, a “stock” of recombinant AAV vector refers to a population of recombinant AAV vector. Despite heterogeneity in their capsid proteins due to deamidation, recombinant AAV in a stock are expected to share an identical vector genome. A stock can include recombinant AAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected.

It should be understood that the compositions in the recombinant AAV vectors described herein are intended to be applied to other compositions, regimens, aspects, embodiments, and methods described across the Specification.

### Pharmaceutical Composition

In one aspect, provided herein is a pharmaceutical composition comprising a vector as described herein in a formulation buffer. In certain embodiments, provided is a pharmaceutical composition comprising a recombinant AAV as described herein in a formulation buffer. In certain embodiments, provided is a pharmaceutical composition comprising anti-AAV neutralizing antibodies (e.g., IVIG, polyclonal antibodies, monoclonal antibodies, or combinations thereof), in a formulation buffer.

In certain embodiments, the formulation further comprises a surfactant, preservative, excipients, and/or buffer dissolved in the aqueous suspending liquid. In one embodiment, the buffer is PBS. Suitably, the formulation is adjusted to a physiologically acceptable pH, e.g., in the range of pH 6 to 8; for intravenous delivery, a pH of 6.8 to about 7.2 may be desired. However, other pHs within the broadest ranges and these subranges may be selected for other route of delivery.

A suitable surfactant, or combination of surfactants, may be selected from among non-ionic surfactants that are nontoxic. In certain embodiments, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly (propylene oxide)) flanked by two hydrophilic chains of polyoxyethylene (poly (ethylene oxide)), SOLUTOL HS 15 (Macrogol-15 Hydroxystearate), LABRASOL (Caprylocaproyl macrogol glycerides), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

Additionally provided is a pharmaceutical composition comprising a pharmaceutically acceptable carrier and a vector comprising a nucleic acid sequence encoding a functional gene product as described herein. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. Delivery vehicles such as liposomes, nanocapsules, microparticles, microspheres, lipid particles, vesicles, and the like, may be used for the introduction of the compositions of the present invention into suitable host cells. In particular, the recombinant AAV vector may be formulated for delivery either encapsulated in a lipid particle, a liposome, a vesicle, a nanosphere, or a nanoparticle or the like. In one embodiment, a therapeutically effective amount of said vector is included in the pharmaceutical composition. The selection of the carrier is not a limitation of the present invention. Other conventional pharmaceutically acceptable carrier, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The phrase “pharmaceutically-acceptable” refers to molecular entities and compositions that do not produce an allergic or similar untoward reaction when administered to a host.

As used herein, the term “dosage” or “amount” can refer to the total dosage or amount delivered to the subject in the course of treatment, or the dosage or amount delivered in a single unit (or multiple unit or split dosage) administration.

Also, the replication-defective virus compositions can be formulated in dosage units to contain an amount of replication-defective virus that is in the range of about 1.0×10⁹ GC to about 1.0×10¹⁶ GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range. In one embodiment, the pharmaceutical composition comprising a rAAV as described herein is administrable at a dose of about 1×10⁹ GC per gram of brain mass to about 1×10¹⁴ GC per gram of brain mass.

In certain embodiments, a pharmaceutical composition comprising a viral vector is formulated for delivery via intracerebroventricular (ICV), intrathecal (IT), or intracisternal injection.

In certain embodiments, the composition comprising the anti-vector neutralizing antibodies (e.g., anti-AAV NAb) is designed for delivery to a subject in need thereof by intravenous injection. Alternatively, other routes of administration may be selected (e.g., subcutaneous, oral, inhalation, intranasal, intratracheal, intraarterial, intraocular, intramuscular, and other parenteral routes).

In certain embodiments, the aqueous suspension or the pharmaceutical composition is used in the preparation of a medicament or kit. In certain embodiments, uses of the same for reducing levels of neutralizing antibodies to a vector (e.g., parental AAV capsid source) in a patient in a need thereof are provided.

It should be understood that the compositions in the pharmaceutical compositions described herein are intended to be applied to other compositions, regimens, aspects, embodiments, and methods described across the Specification.

In certain embodiments, a combination regimen provided herein further comprises co-administering one or more of: (a) a steroid or combination of steroids: (b) an IgG-cleaving enzyme; (c) an inhibitor of Fc-IgE binding; (d) an inhibitor of Fc-IgM binding; (e) an inhibitor of Fc-IgA binding; or (f) gamma interferon.

In certain embodiments, the anti-viral vector construct (e.g., an anti-AAV antibody) is delivered systemically, e.g., intravenously, intraperitoneally, intranasally, or via inhalation.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

A nucleic acid refers to a polymeric form of nucleotides and includes RNA. mRNA. cDNA, genomic DNA, peptide nucleic acid (PNA) and synthetic forms and mixed polymers of the above. A nucleotide refers to a ribonucleotide, deoxynucleotide or a modified form of either type of nucleotide (e.g., a peptide nucleic acid oligomer). The term also includes single- and double-stranded forms of DNA. The skilled man will appreciate that functional variants of these nucleic acid molecules are also intended to be a part of the present invention. Functional variants are nucleic acid sequences that can be directly translated, using the standard genetic code, to provide an amino acid sequence identical to that translated from the parental nucleic acid molecules.

Methods for engineering nucleic acids and sequences are known and have been described previously (e.g., WO 96/09378). A sequence is considered engineered if at least one non-preferred codon as compared to a wild type sequence is replaced by a codon that is more preferred. Herein, a non-preferred codon is a codon that is used less frequently in an organism than another codon coding for the same amino acid, and a codon that is more preferred is a codon that is used more frequently in an organism than a non-preferred codon. The frequency of codon usage for a specific organism can be found in codon frequency tables, such as in www.kazusa.jp/codon. Preferably more than one non-preferred codon, preferably most or all non-preferred codons, are replaced by codons that are more preferred. Preferably the most frequently used codons in an organism are used in an engineered sequence. Replacement by preferred codons generally leads to higher expression. It will also be understood by a skilled person that numerous different nucleic acid molecules can encode the same polypeptide as a result of the degeneracy of the genetic code. It is also understood that skilled persons may, using routine techniques, make nucleotide substitutions that do not affect the amino acid sequence encoded by the nucleic acid molecules to reflect the codon usage of any particular host organism in which the polypeptides are to be expressed. Therefore, unless otherwise specified, a “nucleic acid sequence encoding an amino acid sequence” includes all nucleotide sequences that are degenerate versions of each other and that encode the same amino acid sequence. Nucleic acid sequences can be cloned using routine molecular biology techniques, or generated de novo by DNA synthesis, which can be performed using routine procedures by service companies having business in the field of DNA synthesis and/or molecular cloning (e.g., GeneArt, GenScript, Life Technologies, Eurofins).

The term “percent (%) identity”, “sequence identity.”, “percent sequence identity”, or “percent identical” in the context of nucleic acid sequences refers to the residues in the two sequences which are the same when aligned for correspondence. The length of sequence identity comparison may be over the full-length of the genome, the full-length of a gene coding sequence, or a fragment of at least about 500 to 5000 nucleotides, is desired. However, identity among smaller fragments, e.g., of at least about nine nucleotides, usually at least about 20 to 24 nucleotides, at least about 28 to 32 nucleotides, at least about 36 or more nucleotides, may also be desired.

Multiple sequence alignment programs are also available for nucleic acid sequences. Examples of such programs include, “Clustal Omega”, “Clustal W”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference.

Percent identity may be readily determined for amino acid sequences over the full-length of a protein, polypeptide, about 32 amino acids, about 330 amino acids, or a peptide fragment thereof or the corresponding nucleic acid sequence coding sequences. A suitable amino acid fragment may be at least about 8 amino acids in length, and may be up to about 700 amino acids. Generally, when referring to “identity.”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Unless otherwise specified by an upper range, it will be understood that a percentage of identity is a minimum level of identity and encompasses all higher levels of identity up to 100% identity to the reference sequence. Unless otherwise specified, it will be understood that a percentage of identity is a minimum level of identity and encompasses all higher levels of identity up to 100% identity to the reference sequence. For example. “95% identity” and “at least 95% identity” may be used interchangeably and include 95, 96, 97, 98, 99 up to 100% identity to the referenced sequence, and all fractions therebetween.

Unless otherwise specified, numerical values will be understood to be subject to conventional mathematic rounding rules.

Identity may be determined by preparing an alignment of the sequences and through the use of a variety of algorithms and/or computer programs known in the art or commercially available (e.g., BLAST, ExPASy: Clustal Omega: FASTA: using, e.g., Needleman-Wunsch algorithm, Smith-Waterman algorithm). Alignments are performed using any of a variety of publicly or commercially available Multiple Sequence Alignment Programs. Sequence alignment programs are available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al. Nucl. Acids. Res., “A comprehensive comparison of multiple sequence alignments”, 27 (13): 2682-2690 (1999).

The aqueous suspension or pharmaceutical compositions described herein are designed for delivery to subjects in need thereof by any suitable route or a combination of different routes.

As used herein, the terms “intrathecal delivery” or “intrathecal administration” refer to a route of administration for drugs via an injection into the spinal canal, more specifically into the subarachnoid space so that it reaches the cerebrospinal fluid (CSF). Intrathecal delivery may include lumbar puncture, intraventricular, suboccipital/intracisternal, and/or C1-2 puncture. For example, material may be introduced for diffusion throughout the subarachnoid space by means of lumbar puncture. In another example, injection may be into the cisterna magna. Intracisternal delivery may increase vector diffusion and/or reduce toxicity and inflammation caused by the administration. See, e.g., Christian Hinderer et al, Widespread gene transfer in the central nervous system of cynomolgus macaques following delivery of AAV9 into the cisterna magna, Mol Ther Methods Clin Dev. 2014; 1: 14051. Published online 2014 Dec. 10. In certain embodiments, the intrathecal administration is performed as described in US Patent Publication No. 2018-0339065 A1, published Nov. 29, 2019, which is incorporated herein by reference in its entirety.

As used herein, the terms “intracisternal delivery” or “intracisternal administration” refer to a route of administration for drugs directly into the cerebrospinal fluid of the brain ventricles or within the cisterna magna cerebellomedularis, more specifically via a suboccipital puncture or by direct injection into the cisterna magna or via permanently positioned tube. In one aspect, the vectors, rAAV or compositions thereof provided herein may be administered intrathecally via the method and/or the device provided in this section and described in WO 2017/136500 and WO 2018/160582, which are incorporated by reference herein. Alternatively, other devices and methods may be selected. In certain embodiments, the method comprises the steps of CT-guided sub-occipital injection via spinal needle into the cisterna magna of a patient. As used herein, the term Computed Tomography (CT) refers to radiography in which a three-dimensional image of a body structure is constructed by computer from a series of plane cross-sectional images made along an axis. In certain embodiments, the apparatus is described in US Patent Publication No. 2018-0339065 A1, published Nov. 29, 2019, which is incorporated herein by reference in its entirety. In certain embodiments, the vectors, rAAV or compositions thereof provided herein may be administered using Ommaya Reservoir.

As used herein, the term “intraparenchymal (dentate nucleus)” or “IDN” refers to a route of administration of a composition directly into dentate nuclei. IDN allows for targeting of dentate nuclei and/or cerebellum. In certain embodiments, the IDN administration is performed using ClearPoint® Neuro Navigation System (MRI Interventions, Inc., Memphis, TN) and ventricular cannula, which allows for MRI-guided visualization and administration. Alternatively, other devices and methods may be selected. In one embodiment, a frozen composition is provided which contains an rAAV in a buffer solution as described herein, in frozen form. Optionally, one or more surfactants (e.g., Pluronic F68), stabilizers or preservatives is present in this composition. Suitably, for use, a composition is thawed and titrated to the desired dose with a suitable diluent, e.g., sterile saline or a buffered saline.

“Comprising” is a term meaning inclusive of other components or method steps. When “comprising” is used, it is to be understood that related embodiments include descriptions using the “consisting of” terminology, which excludes other components or method steps, and “consisting essentially of” terminology, which excludes any components or method steps that substantially change the nature of the embodiment or invention. It should be understood that while various embodiments in the specification are presented using “comprising” language, under various circumstances, a related embodiment is also described using “consisting of” or “consisting essentially of” language.

It is to be noted that the term “a” or “an”, refers to one or more, for example, “a vector”, is understood to represent one or more rAAV(s) or another specified vector. As such, the terms “a” (or “an”), “one or more,” and “at least one” is used interchangeably herein.

As used herein, the term “about” means a variability of plus or minus 10% from the reference given, unless otherwise specified.

In certain instances, the term “E+#” or the term “e+#” is used to reference an exponent. For example, “5E10” or “5e10” is 5×10¹⁰. These terms may be used interchangeably.

### EXAMPLES

To test the ability to reduce off-target liver effects, we measured tissue-specific transgene expression in mice and NHPs pretreated with intravenous immunoglobulin (IVIG), which provides NAbs prior to intrathecal AAV administration. IVIG is a purified human IgG pooled from a healthy human population and is commonly used as a therapeutic for a wide range of immune-related diseases. The studies used IVIG having a high neutralizing antibody.

### Example 1: ICV-AAV Results in Peripheral Transgene Expression in Addition to CNS Expression in Mice

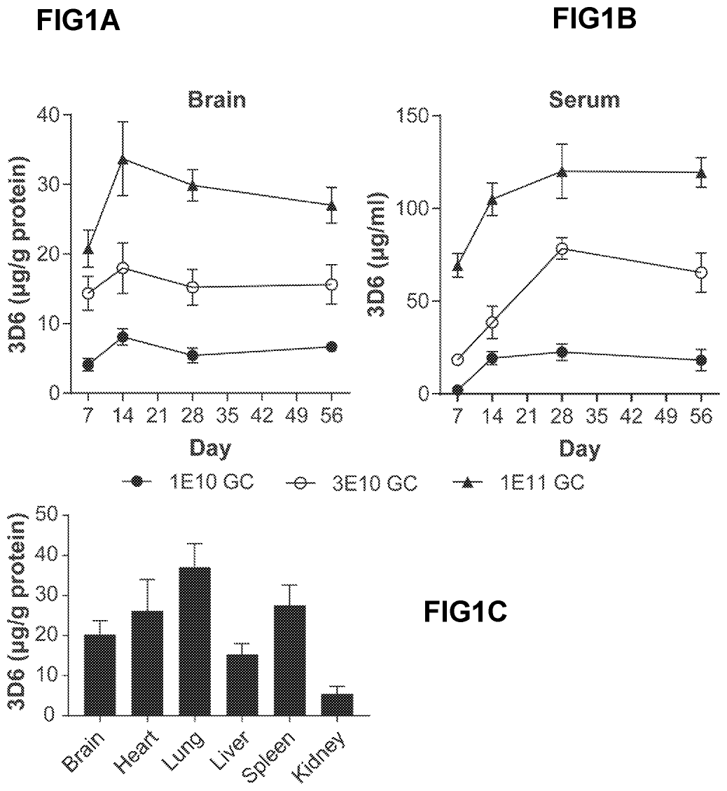

We administered a recombinant AAV serotype hu68 vector expressing a mouse monoclonal antibody (3D6) via unilateral intracerebroventricular (ICV) injection to wild type mice at three doses. Mice were euthanized on days 7, 14, 28, and 56, and 3D6 concentration was measured in brain and serum. 3D6 levels showed a dose-dependent response in both brain and serum. In the brain, 3D6 peaked at day 14 and stayed around 80% of the peak expression up to day 56 (FIG. 1A). In serum. 3D6 accumulated relatively slowly and reached a plateau by day 28 (FIG. 1B). Sustained high levels of serum 3D6 expression suggested the transduction of peripheral organs such as the liver. Consistent with this, 3D6 expression was also detectable in peripheral organs including the heart and liver in mice treated with ICV-AAV at 3e10 GC dose (FIG. 1C). The middle dose, 3e10 GC/mouse, was selected for further studies.

NAb Passive Transfer Prevents Peripheral Organ Transduction while Allowing CNS Transduction of AAVhu68.3D6 in Mice

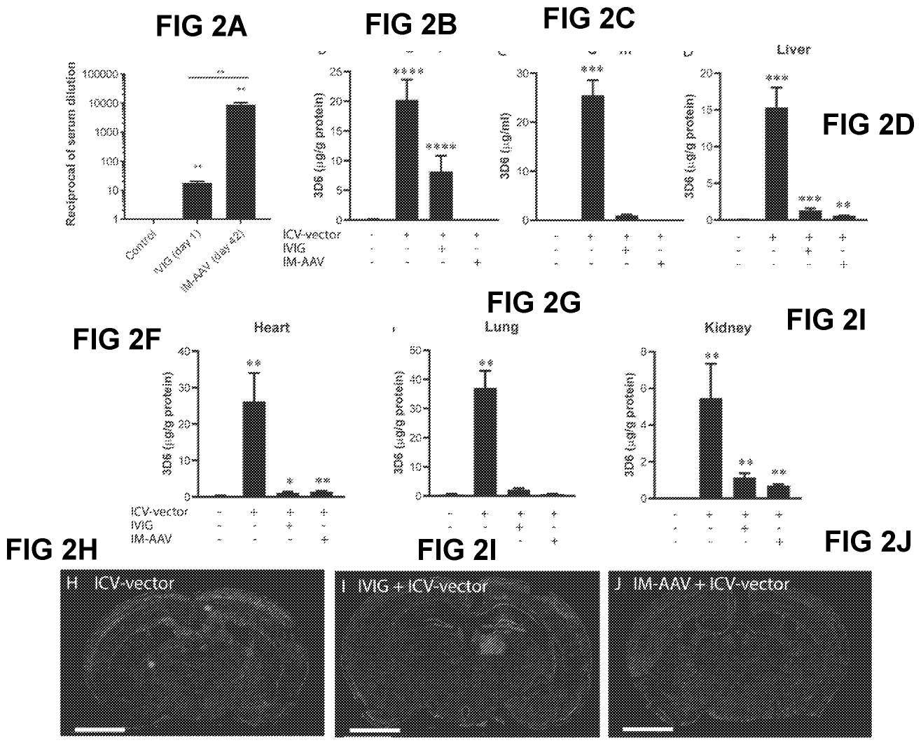

To examine the effect of circulating NAb on intrathecally administered AAV transgene expression, mice were pre-treated with IVIG at 0.5 g/kg 24 h before ICV-AAVhu68.3D6 (IVIG+ICV-vector). The specific IVIG lot used in this study had a 1:1280 AAVhu68 NAb titer in 100 mg/ml solution. Mice that received 1e11 GC AAVhu68.CB7.CI.eGFP.WPRE.rBG intramuscularly at 42 days earlier were also subjected to ICV-AAV as an active immunization group (IM-AAV+ICV-vector). The AAVhu68 neutralization assay indicated that IVIG infusion resulted in a 1:20 AAVhu68 NAb titer at the time of ICV-vector administration, whereas IM AAVhu68.eGFP achieved a NAb titer of 1:10,240 (FIG. 2A). At 28 days after ICV-AAVhu68.3D6, 3D6 was detectable in the brain for the IVIG group, although expression levels were reduced by approximately 50% compared to those in the brains of mice administered ICV-vector without IVIG pre-treatment (ICV-vector). The expression levels of 3D6 in serum and peripheral organs (e.g., liver, heart, lung) were less than 10% of ICV-vector controls in IVIG-pretreated mice. 3D6 was undetectable or very low in brain, serum, and other organs in IM-AAV+ICV-vector group (FIG. 2B-FIG. 2G). In situ hybridization analyses confirmed this expression pattern, in which 3D6 was found to be present in the brain parenchyma of the ICV-vector and IVIG groups but not the IM-AAV group (FIG. 2H-FIG. 2J).

Passive NAb Transfer Prevents ICM-AAV.mAb Transduction to Peripheral Organs while Preserving CNS Transduction in NHPs

We utilized NHPs for further translational studies as their size and anatomy enable the use of the same image-guided ICM injection technique as that employed in clinical trials. These animals were used to test whether IVIG pre-infusion prevented the transduction of peripheral organs while preserving transgene expression in the CNS after ICM-AAV administration. NHPs pre-screened as AAVhu68 NAb<1:5 were infused with 0.5 g/kg IVIG at 24 h prior to ICM-AAV.mAb (AAVhu68.CB7.CI.2.10A.mAb.SV40 at 3e13 GC/animal) (IVIG+ICM-vector group). A simian monoclonal antibody against simian immunodeficiency virus, 2.10A mAb, was used as the transgene in this study. On the day of ICM-AAV administration, the NHPs showed 1:10-1:80 NAb titer, while control NHPs who did not receive IVIG pre-treatment (ICM-vector group) remained at a low NAb titer, <1:5 or 1:5 (Table 1).

TABLE 1
Details of the NHP study groups and AAVhu68 NAb titer at day 0.

| | | | | Body | | AAVhu68 NAb |
|---|---|---|---|---|---|---|
| | | | | weight | AAVhu68 | titer at day 0 |
| | | | | at | NAb | (following IVIG |
| | | | | baseline | titer at | administration for |
| Group | Treatment | Animal ID | Sex | (kg) | baseline | group 2) |
| 1 | AAVhu68.CB7.2.10A.mAb | RA2146 | F | 7.2 | <1:5 | 1:5 |
| | (3 × 10¹³ GC/animal, ICM, day 0) | RA2335 | M | 5.7 | <1:5 | <1:5 |
| | | RA2462 | M | 7.9 | <1:5 | <1:5 |
| | | RA1776 | M | 10.1 | <1:5 | <1:5 |
| 2 | IVIG (0.5 g/kg, IV, day −1) | RA2393 | F | 5.9 | <1:5 | 1:10 |
| | AAVhu68.CB7.2.10A.mAb. | RA2471 | M | 6.3 | <1:5 | 1:10 |
| | (3 × 10¹³ GC/animal, ICM, day 0) | RA2476 | M | 5.7 | <1:5 | 1:20 |
| | | RA1825 | F | 6.2 | <1:5 | 1:80 |

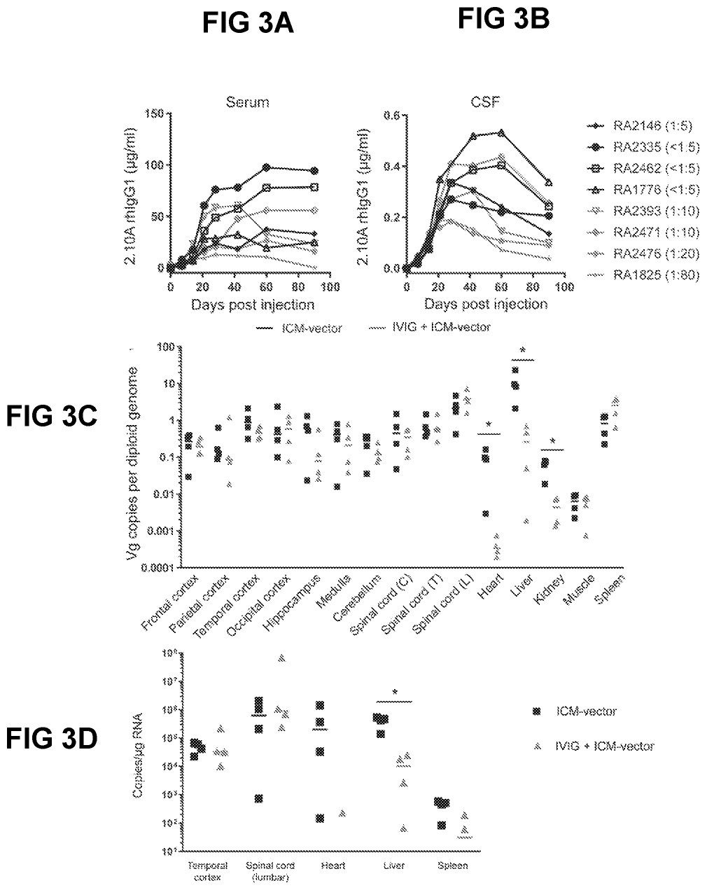

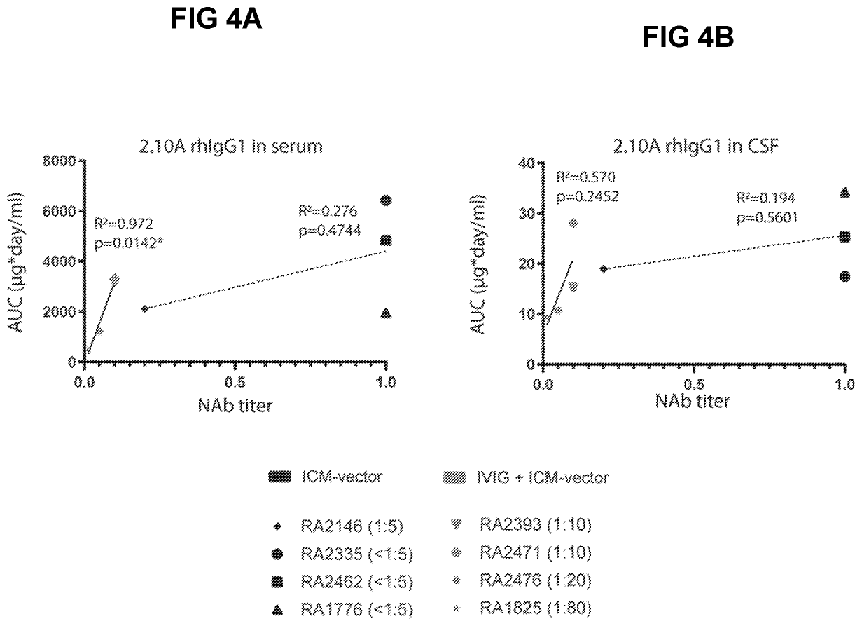

The time course of 2.10A mAb expression in serum (FIG. 3A) and CSF (FIG. 3B) was examined up to 88-91 days post-vector administration (i.e., when all NHPs underwent necropsy). In the ICM-vector group, two NHPs with NAb titer<1:5 showed robust and sustained 2.10A mAb expression in serum whereas the other two NHPs (including one with an increased day 0 NAb titer of 1:5) showed relatively lower serum expression. In the IVIG+ICM-vector group, serum 2.10A mAb expression varied according to day 0 NAb titer. Two NHPs with day 0 NAb titers of 1:20 and 1:80, respectively, showed reduced serum expression, while those with day 0 NAb titers of 1:10 maintained higher expression levels (FIG. 3A). Area under the curve (AUC) analysis highlighted a strong inverse correlation with day 0 NAb titer in IVIG+ICM-vector group (FIG. 41). In CSF, 2.10A mAb level increased similarly among all NHPs in the first 21 days but varied at later time points; NHPs with higher day 0 NAb titers showed relatively lower CSF expression levels. The variable CSF level achieved at later time points could be explained, at least in part, by the differential contribution of serum-derived 2.10A mAb among animals. The AUC showed a moderate inverse correlation with day 0 NAb titer in the IVIG+ICM-vector group (FIG. 3B and FIG. 4B).

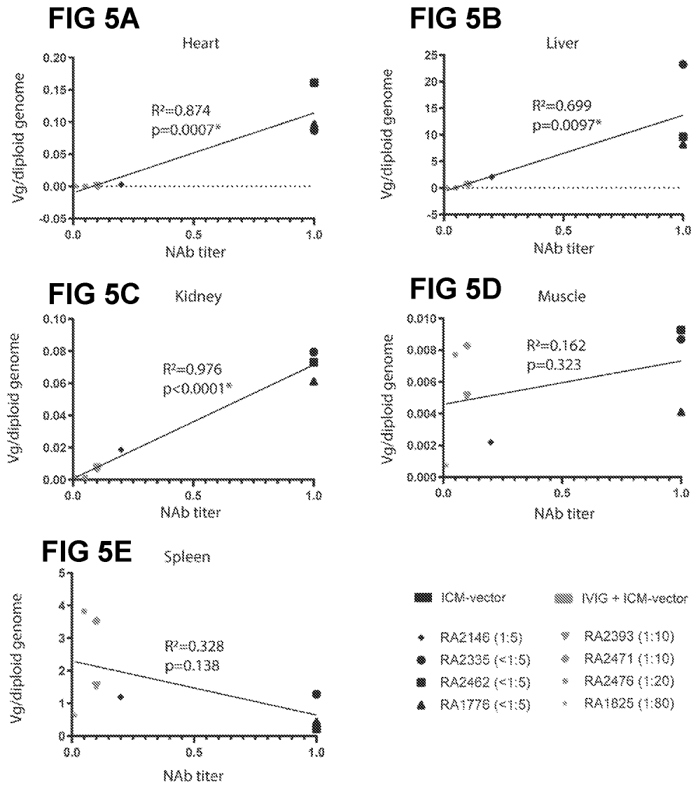

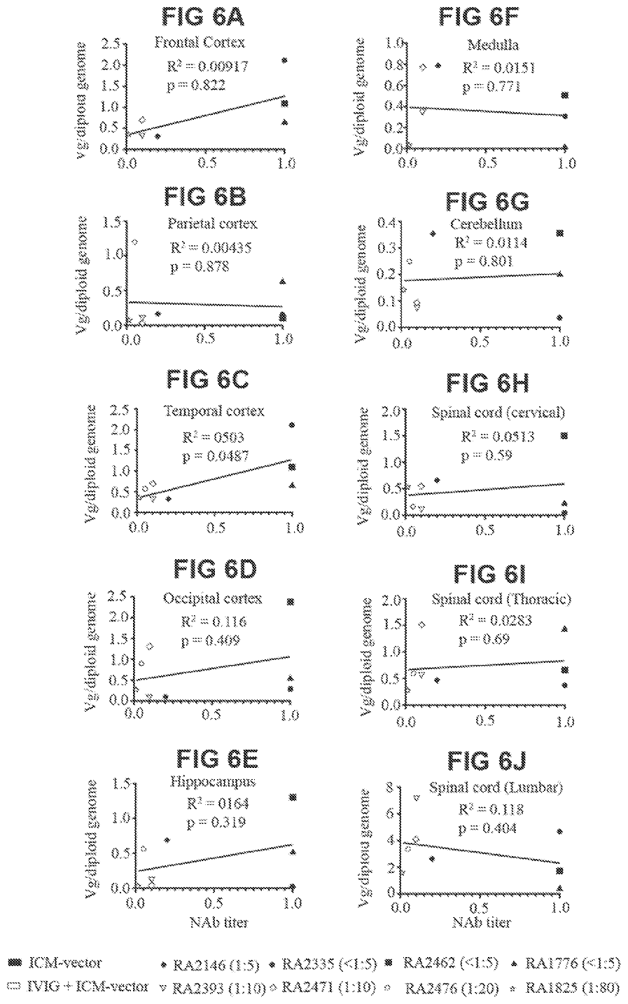

Vector genome biodistribution analysis on tissue samples collected at the necropsy indicated that AAV transduction occurred throughout the CNS to equivalent levels in both the ICM and ICM+IVIG groups, but it was significantly reduced in the heart, liver, and kidney specifically in NHPs pre-treated with IVIG (FIG. 3C). Individual data for IVIG+ICM group shows that transduction to peripheral organs (FIG. 5A-FIG. 5E), including the liver, heart, and kidney, is inversely correlated to day 0 NAb titer, but not in those from the CNS (FIG. 6A-FIG. 6J). Among the peripheral organs examined, skeletal muscle failed to show such a correlation, likely due to its overall low transduction with an ICM-administered vector. On the contrary, the spleen exhibited a positive correlation, which is consistent with our previous finding in pre-existing NAb+NHPs administered intravenous vector that suggested vector may be redirected to this off-target tissue in an antibody-mediated process. [L Wang, Hu Gene Ther, 22, 1389-1401 (2011)]. NHPs with an NAb titer of 1:10 at day 0 showed around 30-fold reduction of liver transduction compared with naïve controls, while those with NAb titers greater than 1:20 showed a greater reduction of 300-fold. Moreover, qRT-PCR analyses highlighted that 2.10A mAb expression in the liver, but not the CNS, was significantly reduced in NHPs pre-treated with IVIG (FIG. 3D). These results are consistent with the hypothesis that 0.5 g/kg IVIG pre-infusion provides anti-AAV NAb that limit vector transduction to peripheral organs, particularly the liver, while preserving CNS transduction after ICM-AAV.

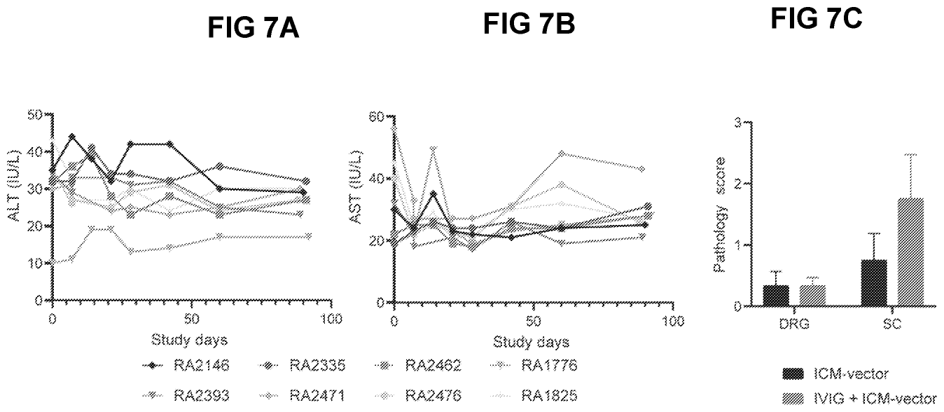

Blood alanine transaminase (ALT) and aspartate transaminase (AST) levels remained within normal ranges during the study in both the ICM and ICM+IVIG groups for the vector dose used in this study (FIG. 7A and FIG. 7B). Clinical pathology analyses of dorsal root ganglia (DRG) indicated that AAV-associated DRG pathology was significantly higher in the ICM+IVIG group, particularly in that observed in the spinal cord (FIG. 7C). Blood cell analysis demonstrated that platelet counts were within the normal range for all NHPs throughout the study except for one NHP in the IVIG group, RA2476 (NAb titer 1:20 at day 0). RA2476 showed a low platelet count (143000 count/μL) at day 7, but this went back to normal by day 14. There were no overall white or red blood cell count deviations from the baseline in any animals, including RA2476. White blood cell counts fluctuated in some NHPs but returned to baseline levels. Histopathology analyses of dorsal root ganglia (DRG) were performed as described previously. Hordeaux, et al, Hum Gene Ther. 31:808-818 (2020). Analysis of pathology scores from all study animals indicated there were no significant differences in AAV-associated DRG pathology levels between groups (FIG. 4C). DRG pathology ranged from normal to grade 1 based on the extend of DRG neuronal degeneration and/or necrosis for both groups. Axonal degeneration in the dorsal spinal white matter showed that more swollen myelin sheaths with axonal debris and myelomacrophages were observed in IVIG+ICM-vector group compared with the ICM-vector group.

This study aimed to evaluate whether passive immunization against AAV vectors reduces liver transduction while preserving CNS transduction upon intrathecal administration of AAV gene therapy in mice (via ICV) and NHPs (via ICM). Our results from both animal models suggest that IVIG pre-treatment represents a method to achieve the clinical objective of improving the safety of CNS-directed AAV gene therapy by reducing adverse effects associated with liver transduction.

In mice, we compared passive immunity transfer via IVIG against active immunization with an intramuscular AAV injection. In situ hybridization data indicated that brain transduction in the IVIG-pretreated groups were comparable to that in the ICV-vector group in mice. However, 3D6 expression appeared to be reduced in the brains of IVIG+ICV-vector mice compared to the ICV-vector group. This likely reflects different methods used to determine 3D6 expression and vector transduction. As circulating IgG can reportedly incorporate into brain endothelial cells and remain in the tissue even after perfusion [Villasenor, et al, Sci Rep, 6:25658 (2016)] peripherally expressed 3D6 in the circulation may have elevated 3D6 protein levels in the brains of the ICV-vector group, as determined in mice by ELISA. IVIG pretreatment prevented peripheral transduction, thereby indirectly reducing 3D6 protein levels in mouse brain by a significant extent. As vector genome qPCR alone was employed to quantify transduction and expression in NHP brains, this reduced expression pattern was not observed.

The passive transfer was successful in reducing liver transduction while permitting CNS transduction, whereas IM-AAV resulted in a much higher NAb titer that completely prevented CNS transduction. These data are consistent with the findings of Wang et al [Wang, D., Zhong, L., Li, M., Li, J., Tran, K., Ren, L., He, R., Xie, J., Moser, R. P., Fraser, C., Kuchel, T., et al. (2018). Adeno-Associated Virus Neutralizing Antibodies in Large Animals and Their Impact on Brain Intraparenchymal Gene Transfer. Mol Ther Methods Clin Dev 11, 65-72. 10.1016/j.omtm.2018.09.003]: the inhibition of CNS transduction in mice with IM-AAV could be due to the resultant extremely high NAb titer and/or pleiotropic immune responses triggered by the active immunization. ICV injection is an invasive procedure which artificially introduces blood components to the CSF, including antibodies. Mccluskey, et al, J Neuroimmunol, 194:27-33 (2008). Although the quantity of antibodies entering CNS is probably limited, a small amount may be sufficient to completely block vector transduction in the context of an extremely high NAb titer. It is known that, unlike in humans, capsid-specific T cells induced by AAV immunization are unable to eliminate transduced hepatocytes with intravenous vector administration in mice. [Li, H., et al, 2007). Pre-existing AAV capsid-specific CD8+ T cells are unable to eliminate AAV-transduced hepatocytes. Mol Ther 15, 792-800. 10.1038/sj_mt_6300090]. However, this could be different with ICV vector administration, which is more invasive and tissue damaging compared to the intravenous route. It was previously demonstrated that ICV, but not ICM, administration induces T cell-mediated encephalitis in unimmunized dogs. [Hinderer, C., et al. (2018). Evaluation of Intrathecal Routes of Administration for Adeno-Associated Viral Vectors in Large Animals. Hum Gene Ther 29, 15-24. 10.1089/hum.2017.026]. In this context, capsid-specific T cells could eliminate transduced brain cells and also contribute to the zero 3D6 expression in mice with active immunization.

The data from NHPs highlight the significant translational potential of IVIG pre-treatment to minimize off-target liver transduction (and associated adverse events) in patients undergoing CNS-targeting AAV gene therapy. ICM vector administration, whose efficacy and safety have been established in NHP pre-clinical studies, is utilized in clinical studies for CNS gene therapies to treat lysosomal storage disorders and frontotemporal dementia. IVIG is a well-tolerated biologic that is widely used in the clinic for patients with immunodeficiencies, autoimmune diseases, and cytokine storms [Alijotas-Reig, J., Esteve-Valverde, E., Belizna, C., Selva-O'Callaghan, A., Pardos-Gea, J., Quintana, A., Mekinian, A., Anunciacion-Llunell, A., and Miro-Mur, F. (2020). Immunomodulatory therapy for the management of severe COVID-19. Beyond the anti-viral therapy: A comprehensive review. Autoimmun Rev 19, 102569. 10.1016/j.autrev.2020.102569]. Passive immunization with IVIG prior to ICM gene therapy could be a viable strategy to improve the safety of current and future CNS-targeting AAV gene therapies if peripheral organ transduction is unwanted. For example, some CNS-targeting gene therapies require a high-level of transgene expression mainly in the CNS tissue but also in the periphery to achieve their therapeutic effect, such as Zolgensma for SMA. In such a case, off-target transduction causing liver-associated toxicity should be avoided. Limiting peripheral expression of CNS-targeted transgenes which may cause toxic effects in peripheral organs should be a priority in the development of safe therapies. For instance, a vectored monoclonal antibody against human epidermal growth factor-2 (HER2) is under development for breast cancer brain metastases [Rothwell, W. T., Bell, P., Richman, L. K., Limberis, M. P., Tretiakova, A. P., Li, M., and Wilson. J. M. (2018). Intrathecal Viral Vector Delivery of Trastuzumab Prevents or Inhibits Tumor Growth of Human HER2-Positive Xenografts in Mice. Cancer Res 78, 6171-6182. 10.1158/0008-5472.CAN-18-0363]: the off-target activity of the antibody on HER2 expressed in the heart is associated with heart failure [Barish, R., Gates, E., and Barac, A. (2019). Trastuzumab-Induced Cardiomyopathy. Cardiol Clin 37, 407-418. 10.1016/j.cel.2019.07.005]. IVIG for NHPs resulted in varied NAb titers ranging 1:10 to 1:80 at day 0. For an ICM-vector dose of 3×10¹³ GC/animal, baseline NAb titers up to 1:80 did not negatively affect CNS transduction. In contrast, transduction of peripheral organs, including the liver, was significantly impaired by IVIG-derived NAb titers as low as 1:20. This is consistent with our previous study in NHPs with intravenous administration of AAV8 vector. [Wang, 2011, Hu Gene Therapy, cited above]. The degree of peripheral organ transduction appeared to vary according to the baseline NAb titer (FIG. 4). These data suggest that higher NAb titers of approximately 1:80 may provide superior protection against adverse liver-associated events caused by off-target peripheral transduction without affecting CNS transduction. However, extremely high NAb titers that can develop as a result of AAV gene therapy (and were observed in mice with active immunization), would likely cause CNS transduction upon ICM vector administration to fail in patients, meaning repeated AAV gene therapy dosing is unlikely to represent a successful approach. This beneficial effect could be particularly useful in optimizing the safety of CNS-targeting gene therapies employing a high ICM AAV vector dose that may cause further off-target liver transduction. To achieve consistently high NAb titers, further improvement in the IVIG dosing regimen with higher doses may be required.

We used the CNS-tropic vector AAVhu68 in this study. The IVIG strategy explored in this study can be theoretically applied to other capsids. However, a limitation of IVIG relates to the fact that it contains different titers of NAb against different capsids [Calcedo, R., Vandenberghe, L. H., Gao, G., Lin, J., and Wilson. J. M. (2009). Worldwide epidemiology of neutralizing antibodies to adeno-associated viruses. J Infect Dis 199, 381-390. 10.1086/595830]. Batch variation in NAb titers may also arise, meaning extensive batch testing and dose adjustment would be required to achieve an appropriate NAb titer on the day of AAV dosing. This strategy could be applied to other routes of vector administration (e.g., intramuscular (IM)) and therefore potentially improve the safety profile of AAV gene therapies directed at target organs other than the CNS. Capsid engineering studies demonstrated promising pre-clinical data of novel liver de-targeting vectors for cardiac and musculoskeletal gene transfer in addition to those selective to the CNS. [Pulicherla, N., Shen, S., Yadav, S., Debbink, K., Govindasamy, L., Agbandje-McKenna, M., and Asokan, A. (2011). Engineering liver-detargeted AAV9 vectors for cardiac and musculoskeletal gene transfer. Mol Ther 19, 1070-1078. 10.1038/mt.2011.22]. Similar to intrathecal administration, IM administration in naïve animals often results in off-target liver transduction (and can cause adverse effects), which is diminished in NAb-positive animals, while transduction of the targeted skeletal muscle is preserved. [Greig, J. A., Calcedo, R., Grant, R. L., Peng, H., Medina-Jaszek, C. A., Ahonkhai, O., Qin, Q., Roy, S., Tretiakova, A. P., and Wilson, J. M. (2016). Intramuscular administration of AAV overcomes pre-existing neutralizing antibodies in rhesus macaques. Vaccine 34, 6323-6329. 10.1016/j.vaccine.2016.10.053]. Activation of complement is involved in adverse events such as inflammation and thrombocytopenia in high-dose systemic AAV gene therapy. [Yazaki, K., Sakuma, S., Hikita, N., Fujimaru, R., and Hamazaki, T. (2022). Child Neurology: Pathologically Confirmed Thrombotic Microangiopathy Caused by Onasemnogene Abeparvovec Treatment for SMA. Neurology 98, 808-813. 10.1212/WNL.0000000000200676]. It is hypothesized that pre-existing anti-AAV antibodies enhance this process by forming immune complexes with AAV vectors and activating the classic complement pathway. [Hinderer, C., Katz, N., Buza, E. L., Dyer, C., Goode, T., Bell, P., Richman, L. K., and Wilson, J. M. (2018). Severe Toxicity in Nonhuman Primates and Piglets Following High-Dose Intravenous Administration of an Adeno-Associated Virus Vector Expressing Human SMN. Hum Gene Ther 29, 285-298. 10.1089/hum.2018.015]. While the introduction of NAb using IVIG could increase this risk, delivering AAV via ICM limits peripheral vector loads compared with systemic AAV delivery, which could reduce AAV-antibody interaction and any resultant complement activation. Our blood work shows that one NHP in the IVIG group, RA2476 (who had a NAb titer 1:20 at day 0), showed a sign of thrombocytopenia at day 7, without changes in white and red blood cell numbers, whereas this was not exhibited by any other NHPs, including RA1825 with NAb=1:80 at day 0. These data make it difficult to conclude whether IVIG contributes to post-AAV complement activation and thrombocytopenia. The DRG pathology finding was unexpected as IVIG itself is safe and commonly used to treat many diseases and conditions including Guillain-Barre syndrome which affects DRG neurons. [Liu, S., Dong, C., and Ubogu, E. E. (2018). Immunotherapy of Guillain-Barre syndrome. Hum Vaccin Immunother 14, 2568-2579. 10.1080/21645515.2018.1493415]. Further investigation and monitoring, including that of intravenous vector groups, are required to address this issue as well as other safety concerns.

Collectively, our data indicate that NAb passive transfer by IVIG, a well-established clinical product, reduces off-target liver transduction when AAV vector is injected intrathecally without affecting CNS transduction. Further refinement of this method will hopefully help to improve the safety of (CNS-targeting) AAV gene therapies in the clinical setting.

### Materials and Methods

### Vectors

3D6 and 2.10A mAbs were cloned into an expression construct flanked by AAV2 inverted terminal repeats containing a chicken beta-actin promoter with a cytomegalovirus early enhancer, chimeric intron, and rabbit beta globin polyA sequence. AAVhu68 vectors were generated via triple transfection of HEK293 cells and iodixanol purification as previously described [Lock, M., Alvira, M., Vandenberghe, L. H., Samanta, A., Toelen, J., Debyser, Z., and Wilson, J. M. (2010). Rapid, simple, and versatile manufacturing of recombinant adeno-associated viral vectors at scale. Hum Gene Ther 21, 1259-1271. 10.1089/hum.2010.055].

### Animal Procedures

All animal procedures were approved by the Institutional Animal Care and Use Committee of the University of Pennsylvania. 6-8-week-old C57BL/6J female mice purchased from the Jackson Laboratory (stock #000664) were used in this study. Privigen® (CLS Behring) was used as IVIG and administrated to mice via the tail vein for NAb passive transfer. For the active immunization of AAV, AAVhu68.CB7.CI.eGFP.WPRE.rBG vector (an rAAV vector having an AAVhu68 capsid and a vector genome comprising a green fluorescent protein expressed under the control of a CB7 promoter (CMV IE early enhancer, spacer sequences and a chicken beta actin promoter), and having a woodchuck post-regulatory control element and a rabbit beta globin polyA) (was administered intramuscularly to gastrocnemius for both sides at 5e10 GC in 25 μL per side. ICV injection was performed with the freehand technique previously described [Hinderer, C., Nosratbakhsh, B., Katz, N., and Wilson, J. M. (2020). A Single Injection of an Optimized Adeno-Associated Viral Vector into Cerebrospinal Fluid Corrects Neurological Disease in a Murine Model of GM1 Gangliosidosis. Hum Gene Ther 31, 1169-1177. 10.1089/hum.2018.206]. Mice were euthanized by exsanguination/cardiac perfusion with DPBS while mice were deeply anesthetized with isoflurane delivered via a facemask at the study endpoint. Eight 4- to 6-year-old rhesus macaques were purchased from Covance Research Products. Four animals for IVIG group were administered Privigen® (CSL Behring) at 0.5 g/kg intravenously at study day-1. At day 0, all animals received a single ICM injection of 3e13 GC of AAVhu68.CB7.CI.2.10A.mAb.SV40 vector in 1 mL of artificial CSF with the fluoroscope image guidance as described previously [Katz, N., Goode, T., Hinderer, C., Hordeaux, J., and Wilson, J. M. (2018). Standardized Method for Intra-Cisterna Magna Delivery Under Fluoroscopic Guidance in Nonhuman Primates. Hum Gene Ther Methods 29, 212-219. 10.1089/hgtb.2018.041].

### ELISA

3D6 mAb in serum or tissue homogenates was measured by sandwich ELISA using the antigen, amyloid-β 1-42 peptide (Abcam, ab120301), and HRP-conjugated anti-mouse IgG antibody for capture and detection, respectively. For 2.10A mAb, the antigen, gp120 (SF162) (Clade B) protein (Immune Technology Corp., IT-001-0028p), was used as the capture protein. The combination of biotin-conjugated goat anti-human IgG (Jackson Immunoresearch, 109-065-098) and HRP-conjugated streptavidin (Abcam, ab7403) was used for detection. Plates coated with the capture protein were blocked, incubated with diluted samples, and then detector reagents with wash steps in-between. HRP activity in each well was developed with TMB substrate that was followed by O.D. 450 nm measurement by Spectramax M3 plate reader (Molecular devices).

Vector Genome and Transgene mRNA Biodistribution

NHP tissue samples were snap frozen at the time of necropsy. DNA and RNA were extracted with QIAamp DNA Mini Kit (Qiagen, 56304) and RNeasy Mini kit (Qiagen. 74104), respectively. Vector genome was measured by real-time PCR using TaqMan assay for SV40 poly A sequence as described previously. RNA was reverse-transcribed into cDNA using High-Capacity cDNA Reverse Transcription kit (Thermo Fisher, 4368814) and 2.10A mAb cDNA was quantified by real-time PCR with the custom TaqMan assay for the transgene.

### NAb Assay

We evaluated neutralizing antibodies against AAVhu68 as previously described [Calcedo, 2009, cited above].

### Clinical Analysis

Blood liver enzymes, AST and ALT, were measured by laboratory diagnostic service by Antech. DRG pathology was evaluated and scored on hematoxylin and eosin (H&E)-stained DRG and spinal cord sections as described previously [Hordeaux, J., et al. (2020). Adeno-Associated Virus-Induced Dorsal Root Ganglion Pathology. Hum Gene Ther 31, 808-818].

### In Situ Hybridization

Mouse brains were fixed in 10% formalin solution, paraffin embedded, sectioned, and subjected to in situ hybridization. ViewRNA ISH Tissue Assay Kit (Thermo Fisher) was used according to the manufacturer's instruction using a probe specifically designed to the codon-optimized 3D6. Bound probes were detected by the Fast Red precipitation. Sections were counter stained with DAPI to show nuclei.

### Statistical Analysis

All quantitative data sets were analyzed using Wilcoxon rank-sum test with function “wilcox.test” within the R Program (version 4.0.0; https://cran.r-project.org). The Benjamini-Hochberg procedure was applied to correct for multiple hypothesis testing. Statistical significance was assessed at the 0.05 level after multiple testing adjustments.

### Example 2: Pretreatment to Block Systemic Delivery of CNS-Delivered AAV

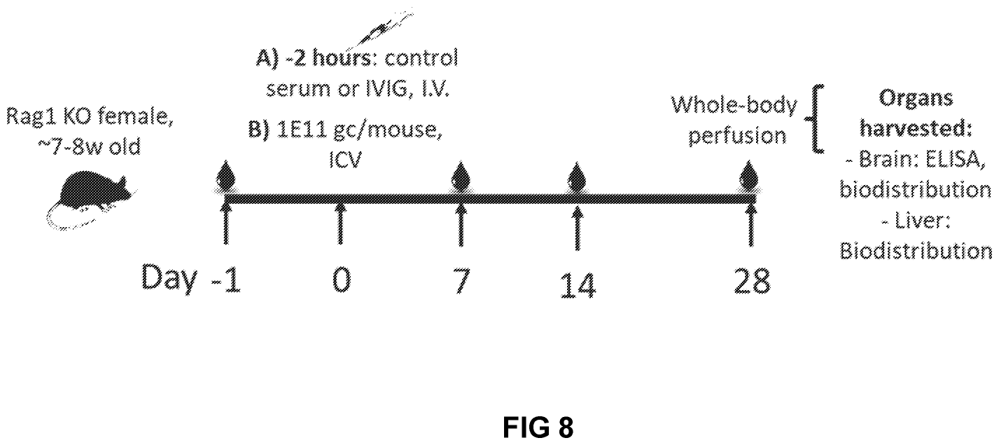

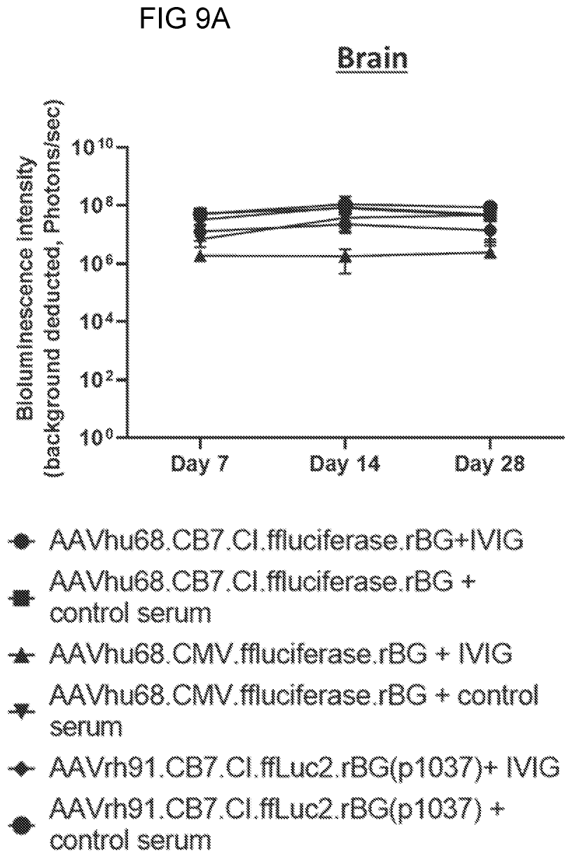

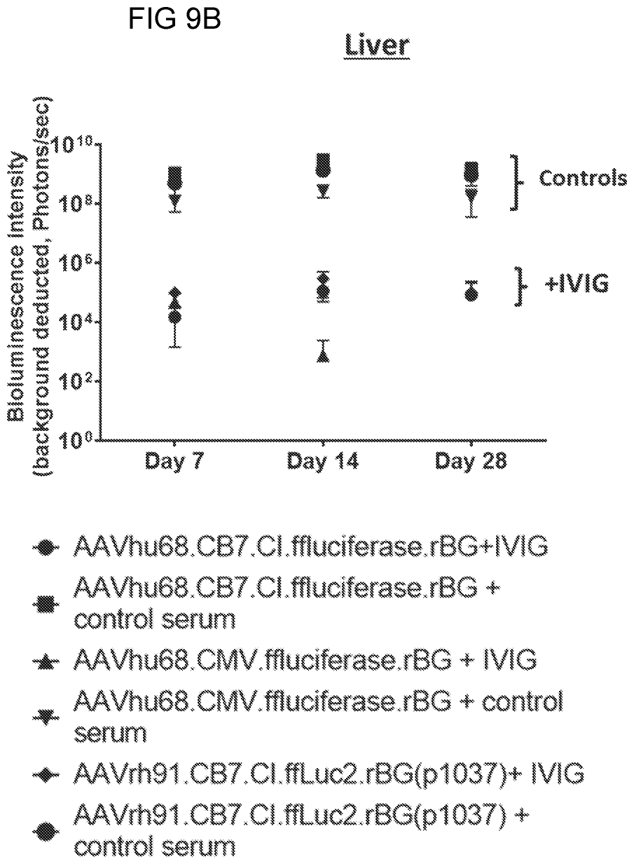

To mitigate the risk of transgene-induced cardiotoxicity in future clinical trials, the use of plasma-derived, pooled human immunoglobulin (IVIG) as a source of anti-AAV neutralizing antibodies to reduce peripheral transduction resulting from vector leakage into the systemic circulation was investigated. In this study (FIG. 8), we pre-treated mice with at 2 hours before treatment (−2) with IVIG via intravenous injection to reduce serum levels of transgene expression, following which mice were administered with rAAVrh91.testgene expressed under a constitutive promoter. On day 28 mice were euthanized, whole body perfusion was performed, and brain and liver tissue samples were collected. FIG. 9A shows expression levels in collected brain tissue following ICV injection with and without IVIG, plotted as bioluminescence intensity in photons/sec (luciferase) on day 7, 14, and 28. FIG. 9B shows expression levels in collected liver tissue following ICV injection with and without IVIG, plotted as bioluminescence intensity in photons/sec (luciferase) on day 7, 14, and 28.

All publications cited in this specification are incorporated herein by reference in their entireties, as is U.S. Provisional patent application No. 63/328,227, filed Apr. 6, 2022. While the invention has been described with reference to particular embodiments, it will be appreciated that modifications can be made without departing from the spirit of the invention. Such modifications are intended to fall within the scope of the appended claims.

1. A combination regimen for preventing systemic uptake of a recombinant adeno-associated virus (AAV) vector delivered to a human central nervous system (CNS) or intraocularly, the regimen comprising (a) pretreating the patient by systemically administering a composition comprising anti-AAV capsid neutralizing antibodies directed against an AAV capsid of the recombinant AAV vector, and (b) administering to the CNS or intraocularly a recombinant AAV vector comprising the AAV capsid and a vector genome comprising a nucleic acid sequence encoding a gene product operably linked to regulatory sequences that direct expression of the gene product in a target cell in the CNS or in the eye.

2. The combination regimen according to claim 1, wherein the pretreating comprises intravenously administering the composition comprising polyclonal antibodies.

3. The combination regimen according to claim 1, wherein the composition comprises pooled immunoglobulin from patients having high anti-AAV titers.

4. The combination regimen according to claim 1, wherein the composition comprises at least one anti-AAV monoclonal antibody.

5. The combination regimen according to claim 1, wherein the composition comprises a cocktail of anti-AAV monoclonal antibodies.

6. The combination regimen according to claim 1, wherein the pretreating occurs at least two hours before and/or up to five days prior to administration of the recombinant AAV vector.

7. The combination regimen according to claim 1, wherein the composition comprising the anti-AAV antibodies is administered intravenously.

8. The combination regimen according to claim 1, wherein the recombinant AAV vector is administered intrathecally.

9. The combination regimen according to claim 1, wherein the recombinant AAV vector is administered to the eye, optionally intravitreally, intra-retinally, subretinally, or suprachoroidally.

10. A method for increasing central nervous system transduction of a recombinant AAV-mediated gene therapy, the method comprising:
(a) systemically delivering a composition comprising anti-AAV neutralizing antibodies that bind an AAV capsid of a recombinant AAV vector, and
(b) administering to the CNS of the patient a recombinant AAV vector that comprises the AAV capsid and a vector genome comprising a nucleic acid sequence encoding a gene product operably linked to regulatory sequences that direct expression of the gene product in a target cell in the CNS.

11. An anti-AAV pharmaceutical composition useful for pretreatment a recombinant AAV gene therapy patient, said composition comprising a physiologically compatible aqueous suspension and anti-AAV neutralizing antibodies formulated for systemic delivery to a human patient.

12. The anti-AAV pharmaceutical composition according to claim 11, wherein the anti-AAV neutralizing antibodies are in a dose of about 500 mg to about 2500 mg.

13. The anti-AAV pharmaceutical composition according to claim 11, wherein the anti-AAV neutralizing antibodies comprise polyclonal antibodies.

14. The anti-AAV pharmaceutical composition according to claim 1, wherein the composition comprises pooled immunoglobulin from subjects having high anti-AAV titers.

15. The anti-AAV composition according to claim 1, wherein the composition comprises at least one anti-AAV monoclonal antibody.

16. The anti-AAV composition according to claim 1, wherein the composition comprises a cocktail of anti-AAV monoclonal antibodies.

17. (canceled)

18. An anti-AAV composition according to claim 1, wherein the composition is to be administered systemically for passive immunization of a patient to reduce off-target transduction of the recombinant AAV in the CNS or in the eye.
